(19) United States
(12) Patent Application Publication
(10) Pub. No.: US 2012/0087914 A1
(43) Pub. Date: Apr. 12, 2012

(54) INHIBITION OF TRIM62 ACTIVITY REDUCES CANCER CELL PROLIFERATION

(75) Inventors: James M. Roberts, Seattle, WA (US); Erik Eide, Seattle, WA (US); Claire Faltermeier, Los Angeles, CA (US)

(73) Assignee: Fred Hutchinson Cancer Research Center

(21) Appl. No.: 12/934,965

(22) PCT Filed: Mar. 25, 2009
(86) PCT No.: PCT/US09/38181

§ 371 Date: Jun. 22, 2011

Related U.S. Application Data
(60) Provisional application No. 61/040,101, filed on Mar. 27, 2008.

(51) Int. Cl. A61K 39/395; A61P 35/00; A61K 31/517; A61K 31/5377; A61K 31/7105; A61K 31/7088

(52) U.S. Cl. 424/133.1; 514/44 A; 424/172.1; 514/266.24; 435/29; 435/6.12; 435/7.92; 436/501; 436/94; 514/234.5; 514/266.4; 424/142.1

(57) ABSTRACT

The present invention provides methods to treat cancers using inhibitors of the TRIM62 protein and methods to identify inhibitors and other modulators of the TRIM62 protein. Pharmaceutical compositions that contain an inhibitor of a TRIM62 protein are also provided.

### CROSS-REFERENCES TO RELATED APPLICATIONS

The present invention claims the benefit of priority under 35 U.S.C. §119(e) of U.S. Provisional Application No. 61/040,101, filed Mar. 27, 2008 (Attorney Docket No. 14538A-009200US), the entire content of which is incorporated herein by reference.

### STATEMENT AS TO RIGHTS TO INVENTIONS MADE UNDER FEDERALLY SPONSORED RESEARCH AND DEVELOPMENT

This invention was made with government support under Grant Number CA118043 awarded by the United States National Institutes of Health. The Government has certain rights in the invention.

### FIELD OF THE INVENTION

The present invention provides methods to treat cancers using inhibitors of the TRIM62 protein and methods to identify inhibitors and other modulators of the TRIM62 protein. Pharmaceutical compositions that contain an inhibitor of a TRIM62 protein are also provided.

### BACKGROUND OF THE INVENTION

Cyclin-dependent kinase inhibitor 1B (p27^{Kip1}) is a tumor suppressor protein that inhibits G1 entry by inhibiting activity of cyclin-cyclin dependent kinases (cyclin-CDKs). Unlike other identified tumor suppressor proteins, p27^{Kip1} mutations are rarely found in human cancers. Analysis of p27^{Kip1} function has been analyzed by site-specific mutation of the protein. Mutation of a phosphorylation site (p27 S10A) required for ubiquitin dependent degradation of the protein demonstrated that p27^{Kip1} must be localized to the cell nucleus to perform its tumor suppressive functions. A second p27^{Kip1} mutant, p27 CK−, was generated and had mutation in amino acids required for binding to cyclin, Arg30Ala and Leu32Ala, and an amino acid required for binding to a CDK, Phe62Ala. Besson et al., Genes Dev. 20:47-64 (2006). The p27 CK− protein was localized to the cytoplasm. A p27 CK knock-in mouse had spontaneous tumors in multiple organs, after just six months, indicating that this form of p27^{Kip1} acted as an oncogene. Denicourt et al., Cancer Res. 67:9238-9243 (2007). Because of the dual role of p27^{Kip1} as both tumor suppressor and an oncogene, understanding of the function and regulation of this protein is needed to provide insight into development of cancer and other diseases of cell proliferation. The present invention meets these and other needs.

### BRIEF SUMMARY OF THE INVENTION

In one aspect, the present invention provides a method of treating a cancer in a human subject by administering a therapeutically effective amount of an inhibitor of a tripartite motif-containing 62 (TRIM62) protein that has at least 95% identity to SEQ ID NO: 1 and regulates expression of a cyclin-dependent kinase inhibitor 1B (p27^{Kip1}) protein. The regulation of the p27^{Kip1} protein can be determined using a variety of assays, e.g., a cell based assay which demonstrates that p27^{Kip1} protein levels increase when TRIM62 activity is inhibited. The inhibitor of the TRIM62 protein is, e.g., an siRNA molecule, an antisense molecule, a small organic molecule, or an antibody that specifically binds to the TRIM62 protein.

In one embodiment, the cancer includes cells that overexpress an erythroblastic leukemia viral oncogene homolog 2 (ErbB2) protein. For this type of cancer, treatment can include administration of an inhibitor of the ErbB2 protein, e.g., trastuzumab and lapatinib. Thus, the ErbB2 inhibitor is, e.g., an antibody that specifically binds to the ErbB2 protein, in particular the extracellular domain of ErbB2, or a small molecule that inhibits, e.g., ligand binding, tyrosine kinase activity, or further transduction of a growth signal. In preferred embodiments the cancer the overexpresses ErbB2 is, e.g., breast cancer, ovarian cancer, prostate cancer, or gastric cancer by ErbB2.

In further embodiments, the cancer is any cancer that overexpresses a member of the epidermal growth factor receptor (EGFR) family, e.g., EGFR, ErbB2, ErbB3, or ErbB4. For this type of cancer, treatment can include administration of an inhibitor of the EGFR family member, e.g., an antibody that specifically binds to the EGFR family member protein, in particular the extracellular domain of the EGFR family member, or a small molecule that inhibits, e.g., ligand binding, tyrosine kinase activity, or further transduction of a growth signal by the EGFR family member,

In another embodiment, the inhibitor of the TRIM62 protein is a TRIM62 specific siRNA molecule, for example, SEQ ID NO:5, 6, 7, and 8.

In another aspect the present invention provides a method of identifying a compound that inhibits proliferation of a mammalian cell, by i) contacting a TRIM62 protein or a host cell comprising a TRIM62 protein with a test compound, wherein the TRIM62 protein has at least 95% identity to SEQ ID NO: 1 and regulates expression of a cyclin-dependent kinase inhibitor 1B (p27^{Kip1}) protein; and ii) assaying an activity of the TRIM62 protein or cellular expression of the TRIM62 protein in the presence of the test compound, comparing that value to a control value determined with the test compound, and identifying a difference in activity or expression that indicates that the test compound modulates the activity or expression of the TRIM62 protein, and in that way identifying the compound that inhibits proliferation of the mammalian cell. The test compound can be a TRIM62 specific siRNA, an antisense molecule, a small organic molecule, or an antibody that specifically binds to a TRIM62 protein. Assays for TRIM62 activity can be done using a purified or partially purified TRIM62 protein, a cell extract or an intact mammalian cell. TRIM62 protein or cellular expression levels can be determined in a mammalian cancer cell or an extract from a mammalian cancer cell.

In one embodiment, the activity or cellular expression of the p27Kip1 protein in the mammalian cancer cell or the extract from the mammalian cancer cell is assessed and compared to a control.

Cell based activity or expression assays can be performed in mammalian cancer cells or cell lines that overexpress a member of the epidermal growth factor receptor family, e.g., EGRF, an erythroblastic leukemia viral oncogene homolog 2 (ErbB2), ErbB3, or ErbB4. In a preferred embodiment the cancer cell overexpresses the ErbB2 protein.

In another embodiment, TRIM62 protein or cellular expression levels are assayed in the presence of an inhibitor the ErbB2 protein. Such assays are preferably performed in cells that overexpress the ErbB2 protein or that have high levels of ErbB2 activity.

In another aspect, the present invention provides a method of diagnosing a cancer with increased levels of expression of an epidermal growth factor receptor (EGFR) receptor family member, wherein the cancer is resistant to treatment with a compound that specifically inhibits activity of the EGFR receptor member, by determining the level of expression or activity of a tripartite motif-containing 62 (TRIM62) protein in a sample from the cancer and comparing the TRIM62 protein expression or activity level to a control sample, wherein a difference from the control indicates that the cancer is resistant to treatment with a compound that specifically inhibits activity of the EGFR receptor family member. Assays can include determination of TRIM62 protein or mRNA levels. In one embodiment, activity or expression levels of a cyclin-dependent kinase inhibitor 1B (p27Kip1) protein are also determined in the cancer sample and the control. In a preferred embodiment, the cancer is a breast cancer and the EGFR family member is an erythroblastic leukemia viral oncogene homolog 2 (ErbB2) protein. This type of cell can be resistant to an ErbB2 specific compound, such as lapatinib or trastuzumab.

Another aspect of the present invention provides a pharmaceutical composition that includes a modulator of a tripartite motif-containing 62 (TRIM62) protein, which has at least 95% identity to SEQ ID NO: 1 and regulates expression of a cyclin-dependent kinase inhibitor 1B (p27Kip1) protein. The regulation of the p27^{Kip1} protein can be determined using a variety of assays, e.g., a cell based assay which demonstrates that p27^{Kip1} protein levels increase when TRIM62 activity is inhibited. The inhibitor of the TRIM62 protein is, e.g., an siRNA molecule, an antisense molecule, a small organic molecule, or an antibody that specifically binds to the TRIM62 protein.

In one embodiment, the modulator of the TRIM62 protein inhibits an activity of the TRIM62 protein or cellular expression of the TRIM62 protein. The modulator can be, e.g., an siRNA molecule that inhibits expression of the TRIM62 protein in a host cell. Other possible modulators include, e.g., small organic molecules that inhibit TRIM62 activity and antibodies that specifically bind to the TRIM62 protein. Exemplary TRIM62 specific siRNA sequences include, e.g., SEQ ID NO:5, 6, 7, and 8.

In another embodiment the pharmaceutical composition includes the TRIM62 modulator and an inhibitor of an epidermal growth factor receptor (EGFR) receptor family member. The EGFR family member can be, e.g., the epidermal growth factor receptor (EGFR), an erythroblastic leukemia viral oncogene homolog 2 (ErbB2), ErbB3, and ErbB4. Exemplary inhibitors of EGFR family members include, e.g., trastuzumab lapatinib, gefitinib, erlotinib, cetuximab, panitumumab, pertuzumab, and canertinib. In a preferred embodiment, the EGFR family member is ErbB2 and its inhibitor is trastuzumab or lapatinib.

### BRIEF DESCRIPTION OF THE DRAWINGS

FIGS. 1A and B demonstrate the effect of reducing expression of the human tripartite motif-containing 62 (TRIM62) nucleic acid and protein on the expression of p27^{Kip1} in HeLa cells, a transformed cell line. TRIM62 siRNA was transfected into HeLa cells and transiently expressed. Cell extracts were prepared and proteins were separated using SDS-PAGE. After transfer of the separated proteins to a nylon filter, p27 levels were assayed by Western blotting using a p27^{Kip1} specific antibody. FIG. 1A shows the results with three different TRIM62 siRNA molecules, in lanes 1, 2, and 3. A control sample, labeled mock, was transfected with an unrelated siRNA. Elimination of TRIM62 expression using TRIM62 siRNA's 1 and 3 greatly increased the levels of p27^{Kip1} protein as measured by Western blotting. As a control, levels of the unrelated Grb2 protein were assessed by reprobing the blot with a Grb2 specific antibody.

In FIG. 1B, HeLa cells were treated with cyclohexamide (CXH) over a period of nine hours. Control samples were transfected with an unrelated siRNA. Experimental samples were transfected with a TRIM62 specific siRNA. As a control, levels of the unrelated tubulin protein were assessed by reprobing the blot with a tubulin specific antibody.

FIGS. 2A-C demonstrate the effect of reducing expression of the TRIM62 nucleic acid and protein on the expression of p27^{Kip1} in U251 cells, a transformed cell line derived from a glioblastoma. U251 cells were stably transfected to express a TRIM62 siRNA. Cell extracts were prepared and proteins were separated using SDS-PAGE. After transfer of the separated proteins to a nylon filter, p27^{Kip1} levels were assayed by Western blotting using a p27^{Kip1} specific antibody. FIG. 2A shows the results with a TRIM62 siRNA molecule, in lane 2. A control sample, labeled pBABE-SH-CTL, was transfected with an unrelated siRNA. Elimination of TRIM62 expression using TRIM62 siRNA greatly increased the levels of p27^{Kip1} protein as measured by Western blotting. As a control, levels of the unrelated Grb2 protein were assessed by reprobing the blot with a Grb2 specific antibody.

In FIG. 2B, U251 cells were treated with cyclohexamide (CXH) over a period of six hours. Control samples (mock) were transfected with an unrelated siRNA. Experimental samples were transfected with a TRIM62 specific siRNA. As a control, levels of the unrelated tubulin protein were assessed by reprobing the blot with a tubulin specific antibody. FIG. 2C is a graph showing the time course of p27^{Kip1} expression after CHX treatment in the presence and absence of a TRIM62 siRNA.

FIGS. 3A and 3B show the effect of serum readdition on p27^{Kip1} levels after a G1 cell cycle block on breast cancer cell lines that do not overexpress the ErbB2 protein. p27^{Kip1} levels were assayed by Western blotting using a p27^{Kip1} specific antibody. FIG. 3A shows the effect of serum readdition on p27^{Kip1} protein expression after serum starvation. FIG. 3B shows a time course of p27^{Kip1} protein levels during a twenty-four hour period following readdition of serum to serum-starved cells.

FIGS. 4A and 4B show the effect of serum readdition on p27^{Kip1} levels after a G1 cell cycle block on five breast cancer cell lines that have an amplification of the ErbB2 gene. p27^{Kip1} levels were assayed by Western blotting using a p27^{Kip1} specific antibody. FIG. 4A shows the effect of serum readdition on p27^{Kip1} protein expression after serum starvation. FIG. 4B shows a time course of p27^{Kip1} protein levels during a twenty-four hour period following readdition of serum to serum-starved cells.

FIGS. 5A and B demonstrate the levels of TRIM62 mRNA and p27^{Kip1} mRNA in various human breast cancer cells. RNA levels were assessed in proliferating cells (gray bars) or quiescent cells (black bars). Results for breast cancer cell lines with an amplified ErbB2 gene are boxed at the right side of the graphs. FIG. 5A shows levels of TRIM62 mRNA in breast cancer cells and FIG. 5B shows levels of p27^{Kip1} mRNA in breast cancer cells.

FIG. 6 demonstrates that TRIM62 protein regulates p27^{Kip1} in many breast cancer cell lines. Breast cancer cells with unmodified ErbB2 expression (MDA231, MCF7, and HCC38) and breast cancer cells with amplification of the ErbB2 gene (UAC893 and HCC1569) were assessed. Cells were transfected with control siRNA, TRIM62 siRNA or Skp2 siRNA and levels of p27^{Kip1} protein were determined by western blotting with a p27^{Kip1} specific antibody.

FIG. 7 demonstrates that elimination of TRIM62 activity enhances increased p27 expression in the presence of lapatinib, an antibody specific for the ErbB2 protein. A breast cancer cell line with an amplified ErbB2 gene (UACC893) was transfected with a TRIM62 siRNA expression vector or a vector that expressed a control siRNA. p27^{Kip1} levels were assayed by Western blotting using a p27^{Kip1} specific antibody. After treatment of the control-transfected cells with 0.5 μM lapatinib, p27^{Kip1} levels increased as compared to the untreated cells. Cells transfected with the TRIM62 siRNA also had increased p27^{Kip1} levels. The highest levels of p27^{Kip1} expression were seen in cells that were transfected with TRIM62 siRNA and treated with lapatinib.

FIG. 8 demonstrates that TRIM62 overexpression prevents lapatinib-dependent p27^{Kip1} accumulation in breast cancer cells with an amplified ErbB2 gene. A breast cancer cell line with an amplified ErbB2 gene (UACC893) was transfected with a TRIM62 expression vector or an empty vector. p27^{Kip1} levels were assayed by Western blotting using a p27^{Kip1} specific antibody. Cells were treated with 0.5 μM lapatinib or buffer.

FIG. 9 provides an assessment of TRIM62siRNA on the cell cycle of MCA10F exponentially growing cells. The cells were sorted on siGLO-FITC. TRIM62siRNA demonstrates that a higher percentage of the MCA10F cells remain in G1 subsequent to treatment.

### DETAILED DESCRIPTION OF THE INVENTION

### I. Introduction

This invention provides, for the first time, evidence that expression of the p27^{Kip1} protein is regulated by the activity of the TRIM62 protein, a ubiquitin E3 ligase. When localized to the nucleus, the p27^{Kip1} protein inhibits the activity of cyclin-CDK proteins and inhibits the cell cycle in G1. Inactivation of the TRIM62 protein by, e.g., administration of an siRNA specific for TRIM62 nucleic acid, resulted in increased expression of the p27^{Kip1} protein in cells, including all tested cells derived from human cancers. Thus, compounds that modulate cell proliferation are discovered by identification of compounds that modulate TRIM62 activity. In a preferred embodiment, compounds that inhibit cell proliferation are discovered by identification of compounds that inhibit TRIM62 activity. Similarly, compounds that modulate p27^{Kip1} protein activity or expression are discovered by identification of compounds that modulate TRIM62 activity. In a preferred embodiment, compounds that increase p27^{Kip1} protein activity or expression are discovered by identification of compounds that inhibit TRIM62 activity. Finally, compounds that inhibit TRIM62 activity are used to treat cancer, preferably in a human subject.

Certain cancers have increased expression or activity of members of the epidermal growth factor receptor (EGFR) family. Many of these cancers can be treated using therapeutics that specifically inactivate the EGFR family member. p27^{Kip1} protein accumulates in transformed cells that have increased activity or expression of a member of the epidermal growth factor receptor family, e.g., an ErbB2 amplification. Administration of TRIM62 siRNA to, e.g., ErbB2 amplified, cells led to further enhancement of p27^{Kip1} protein expression. Thus, compounds that inhibit proliferation of transformed cells with increased activity or expression of a member of the EGFR family are discovered by identification of compounds that inhibit TRIM62 activity. In a preferred embodiment, the transformed cell has an amplified ErbB2 gene. Similarly, compounds that inhibit p27^{Kip1} protein activity or expression in transformed cells with increased activity or expression of a member of the EGFR family are discovered by identification of compounds that modulate TRIM62 activity. In a preferred embodiment, the transformed cell has an amplified ErbB2 gene. Finally, compounds that inhibit TRIM62 activity are used to treat a cancer that includes cells that have increased expression or activity of an EGFR family member, preferably in a human subject. In some embodiments, the cancer is treated by administration of a combination of a compound that specifically targets the EGFR family member and a compound that inhibits TRIM62 activity. In a preferred embodiment, the cancer has an amplification of the ErbB2 gene and is treated by administration of an inhibitor of TRIM62 activity. In a further preferred embodiment, the cancer is treated by administration of a compound that specifically targets and inhibits the ErbB2 protein and a compound that inhibits TRIM62 activity.

While some cancers with increased expression or activity of members of the EGFR family respond to therapies that are specifically directed against the EGFR protein, other cancers with increased expression or activity of members of the EGFR family are resistant to such treatment. Identification of such EGFR resistant cancers is useful to formulate an appropriate course of treatment for the subject. Overexpression of the TRIM62 protein in cells with increased expression or activity of members of the EGFR family, such as ErbB2, prevents the accumulation of the p27^{Kip1} protein typically seen after administration of a compound specifically directed against the EGFR family member. For example, overexpression of TRIM62 protein in cancer cells with an ErbB2 amplification prevents accumulation of p27^{Kip1} protein after administration of lapatinib. Thus, measurement of TRIM62 levels is used to identify cancers that are resistant to treatments directed against amplified or overexpressed EGFR family members. Increased levels of TRIM62 protein, as compared to a control, indicates that a cancer is resistant to treatment specifically directed against an EGFR family member. In a preferred embodiment, increased levels of TRIM62 protein, as compared to a control, indicates that a cancer with ErbB2 gene amplification or increased activity is resistant to treatment with an ErbB2 specific agent, e.g., lapatinib or trastuzumab. In another embodiment, cancers that are resistant to agents specifically directed against an EGFR family member and that overexpress the TRIM62 protein are treated with a compound that inhibits activity or expression of the TRIM62 protein.

### II. Definitions

The term “human tripartite motif-containing 62 (TRIM62) protein” or “TRIM62” or grammatical variants, refers to aTRIM62 protein and polypeptide polymorphic variants, alleles, mutants, and interspecies homologs that: (1) have an amino acid sequence that has at least 60% amino acid sequence identity, 65%, 70%, 75%, 80%, 85%, 90%, preferably 91%, 92%, 93%, 94%, 95%, 96%, 97%, 98% or 99% or greater amino acid sequence identity, preferably over a region of at least about 25, 50, 100, 200, 500, 1000, or more amino acids, to an amino acid sequence encoded by a TRIM62 nucleic acid or to an amino acid sequence of a TRIM62 protein (for exemplary TRIM62 protein sequences, see, e.g., SEQ ID NO:1 or accession number NP_{—}060677) and (2) bind to antibodies, e.g., polyclonal antibodies, raised against an immunogen comprising an amino acid sequence of a TRIM62 protein, and conservatively modified variants thereof. The nucleic acids and proteins of the invention include both naturally occurring or recombinant molecules. A TRIM62 protein typically has ubiquitin E3 ligase activity in vitro, and/or activity that regulates the levels of a p27 protein in a cell or animal. Ubiquitin E3 ligase assays can be performed according to methods known to those of skill in the art, using substrates, as described herein and elsewhere. See, e.g., Davydov, et al., Soc. Biomol. Screen. 9:695-703 (2004). In a preferred embodiment, sequence identity is measured over the full length of a reference TRIM62 sequence, e.g., SEQ ID NO:1.

The term “cyclin-dependent kinase inhibitor 1B (p27^{Kip1}) protein” or “p27^{Kip1}” or “p27” or grammatical variants, refers to a p27^{Kip1} protein and polypeptide polymorphic variants, alleles, mutants, and interspecies homologs that: (1) have an amino acid sequence that has at least 60% amino acid sequence identity, 65%, 70%, 75%, 80%, 85%, 90%, preferably 91%, 92%, 93%, 94%, 95%, 96%, 97%, 98% or 99% or greater amino acid sequence identity, preferably over a region of at least about 25, 50, 100, 200, 500, 1000, or more amino acids, to an amino acid sequence encoded by a p27^{Kip1} nucleic acid or to an amino acid sequence of a p27^{Kip1} protein (for exemplary p27^{Kip1} protein sequences, see, e.g., SEQ ID NO:2 or accession number NP_{—}004055) and (2) bind to antibodies, e.g., polyclonal antibodies, raised against an immunogen comprising an amino acid sequence of a p27^{Kip1} protein, and conservatively modified variants thereof. The nucleic acids and proteins of the invention include both naturally occurring or recombinant molecules. A p27^{Kip1} protein typically has cyclin dependent kinase inhibitory activity in vitro, and/or activity that regulates entry into the G1 phase of the cell cycle in a cell or animal. Cyclin dependent kinase assays and measurement of inhibition of that activity can be performed according to methods known to those of skill in the art, using substrates, as described herein and elsewhere. Assays of cell cycle arrest are also known. See, e.g., Polyak, et al., Genes Dev. 8:9-22 (1994). In a preferred embodiment, sequence identity is measured over the full length of a reference p27^{Kip1} sequence, e.g., SEQ ID NO:2.

The term “erythroblastic leukemia viral oncogene homolog 2” or “ErbB2” or grammatical variants, refers to an ErbB2 protein and polypeptide polymorphic variants, alleles, mutants, and interspecies homologs that: (1) have an amino acid sequence that has at least 60% amino acid sequence identity, 65%, 70%, 75%, 80%, 85%, 90%, preferably 91%, 92%, 93%, 94%, 95%, 96%, 97%, 98% or 99% or greater amino acid sequence identity, preferably over a region of at least about 25, 50, 100, 200, 500, 1000, or more amino acids, to an amino acid sequence encoded by an ErbB2 nucleic acid or to an amino acid sequence of an ErbB2 protein (for exemplary ErbB2 proteins sequences, see, e.g., SEQ ID NO:3 and 4 or accession numbers NP_{—}004439 and NP_{—}001005862) and (2) bind to antibodies, e.g., polyclonal antibodies, raised against an immunogen comprising an amino acid sequence of an ErbB2 protein, and conservatively modified variants thereof. The nucleic acids and proteins of the invention include both naturally occurring or recombinant molecules. An ErbB2 protein is a tyrosine kinase and assays of tyrosine kinase activity are well known to those of skill. In a preferred embodiment, sequence identity is measured over the full length of a reference ErbB2 sequence, e.g., SEQ ID NO:3 or 4.

“Subject” refers to an individual in need of treatment for a particular disease or condition. In a preferred embodiment, the subject is a human in need of treatment for cancer, e.g., for breast cancer treatment.

The phrase “double-stranded ribonucleic acid molecule” or “dsRNA” as used herein refers to any RNA molecule, fragment or segment containing two strands forming an RNA duplex, notwithstanding the presence of single stranded overhangs of unpaired nucleotides. Further, as used herein, a double-stranded ribonucleic acid molecule includes single stranded RNA molecules forming functional stem-loop structures, such as small temporal RNAs, short hairpin RNAs and microRNAs, thereby forming the structural equivalent of an RNA duplex with single strand overhangs. The RNA molecule may be isolated, purified, native or recombinant, and may be modified by the addition, deletion, substitution and/or alteration of one or more nucleotides, including non-naturally occurring nucleotides, also including those added at 5′ and/or 3′ ends to increase nuclease resistance.

The double-stranded ribonucleic acid molecule may be any one of a number of non-coding RNAs (i.e., RNA which is not mRNA, tRNA or rRNA), including, preferably, a small interfering RNA, but may also comprise a small temporal RNA, small nuclear RNA, small nucleolar RNA, short hairpin RNA or a microRNA having either a double-stranded structure or a stem loop configuration comprising an RNA duplex with or without single strand overhangs. The double-stranded RNA molecule may be very large, comprising thousands of nucleotides, or preferably in the case of siRNA protocols involving mammalian cells, may be small, in the range of about 15 to about 25 nucleotides, preferably in the range of about 15 to about 19 nucleotides.

The phrase “small interfering RNA” or “siRNA” as used herein, refers to a double stranded RNA duplex of any length, with or without single strand overhangs, wherein at least one strand, putatively the antisense strand, is homologous to the target mRNA to be degraded. The difference between antisense and double stranded small interfering molecules is that an antisense molecule is a single stranded oligonucleotide which is complementary to a section of the target RNA and must hybridize or bind to it in a 1:1 ratio in order to cause its degradation. In contrast, siRNA provides a substrate for the RNA-induced silencing complex (RISC), and unlike antisense, is inactive until incorporated into this macromolecular complex. This RISC complex is then guided by the unwound siRNA to its target gene. Once the target gene is located, it is destroyed by cleaving the target gene into small pieces, and thereby preventing its expression.

In a preferred embodiment, the siRNA comprises a double-stranded RNA duplex of at least about 15, or preferably at least about 19, nucleotides with no overhanging nucleotides. In another embodiment, the siRNA has nucleotide overhangs. For example, the siRNA may have two nucleotide overhangs, thus the siRNA will comprise a 21 nucleotide sense strand and a 21 nucleotide antisense strand paired so as to have a 19 nucleotide duplex region. The number of nucleotides in the overhang can be in the range of about 1 to about 6 homologous nucleotide overhangs at each of the 5′ and 3′ ends, preferably, about 2-4, more preferably, about 3 homologous nucleotide overhangs at each of the 5′ and 3′ ends. The nucleotide overhang can be modified, for example to increase nuclease resistance. For example, the 3′ overhang can comprise 2′ deoxynucleotides, e.g., TT, for improved nuclease resistance.

“Inhibitors”, “activators”, and “modulators” refer to activating, inhibitory, or modulating molecules identified using in vitro and in vivo assays of, e.g., TRIM62 activity or expression, cellular proliferation, p27 activity or expression, or tumorigenesis; disclosed herein. Inhibitors are compounds that, e.g., bind to, partially or totally block activity, decrease, prevent, delay activation, inactivate, desensitize, or down regulate the activity or expression of a protein, such as TRIM62, or a cell pathway or function, e.g., cell proliferation, protein degradation or tumorigenesis, disclosed herein, e.g., antagonists. “Activators” are compounds that increase, open, activate, facilitate, enhance activation, sensitize, agonize, or up regulate expression or activity of a protein, such as TRIM62, or a cell pathway or function, e.g., cell proliferation, protein degradation or tumorigenesis, disclosed herein, e.g., agonists. Inhibitors, activators, or modulators also include genetically modified versions of, e.g., TRIM62, disclosed herein, e.g., versions with altered activity, as well as naturally occurring and synthetic ligands, antagonists, agonists, antibodies, peptides, cyclic peptides, nucleic acids, antisense molecules, ribozymes, small chemical molecules and the like. Such assays for inhibitors and activators include, e.g., expressing TRIM62 protein disclosed herein in vitro, in cells, or cell membranes or in animals, applying putative modulator compounds, and then determining effects on activity.

Samples or assays comprising a TRIM62 protein disclosed herein that are treated with a potential activator, inhibitor, or modulator are compared to control samples without the inhibitor, activator, or modulator to examine the extent of inhibition. Control samples (untreated with inhibitors) are assigned a relative protein activity value of 100%. Inhibition is achieved when the activity value relative to the control is about 80%, preferably 50%, more preferably 25%. Activation is achieved when the activity value relative to the control (untreated with activators) is 110%, more preferably 150%, more preferably 200-500% (i.e., two to five fold higher relative to the control), more preferably 1000-3000% higher.

The term “test compound” or “drug candidate” or “modulator” or grammatical equivalents as used herein describes any molecule, either naturally occurring or synthetic, e.g., protein, nucleic acid, including dsRNA or siRNA molecules, oligopeptide (e.g., from about 5 to about 25 amino acids in length, preferably from about 10 to 20 or 12 to 18 amino acids in length, preferably 12, 15, or 18 amino acids in length), small organic molecule, polysaccharide, lipid, fatty acid, polynucleotide, oligonucleotide, etc., to be tested for the capacity to directly or indirectly modulation tumor cell proliferation. The test compound can be in the form of a library of test compounds, such as a combinatorial or randomized library that provides a sufficient range of diversity. Test compounds are optionally linked to a fusion partner, e.g., targeting compounds, rescue compounds, dimerization compounds, stabilizing compounds, addressable compounds, and other functional moieties. Conventionally, new chemical entities with useful properties are generated by identifying a test compound (called a “lead compound”) with some desirable property or activity, e.g., inhibiting activity, creating variants of the lead compound, and evaluating the property and activity of those variant compounds. Often, high throughput screening (HTS) methods are employed for such an analysis.

A “small organic molecule” refers to an organic molecule, either naturally occurring or synthetic, that has a molecular weight of more than about 50 daltons and less than about 2500 daltons, preferably less than about 2000 daltons, preferably between about 100 to about 1000 daltons, more preferably between about 200 to about 500 daltons.

“Biological sample” includes sections of tissues such as biopsy and autopsy samples, and frozen sections taken for histologic purposes. Such samples include blood, sputum, tissue, cultured cells, e.g., primary cultures, explants, and transformed cells, stool, urine, etc. A biological sample is typically obtained from a eukaryotic organism, most preferably a mammal such as a primate e.g., chimpanzee or human; cow; dog; cat; a rodent, e.g., guinea pig, rat, mouse; rabbit; or a bird; reptile; or fish.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

Those of skill recognize that many amino acids can be substituted for one another in a protein without affecting the function of the protein, i.e., a conservative substitution can be the basis of a conservatively modified variant of a protein such as the disclosed TRIM62 or p27 proteins and derivatives thereof. An incomplete list of conservative amino acid substitutions follows. The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V), Alanine (A); 6) Phenylalanine (F), Tyrosine (Y), Tryptophan (W); 7) Serine (S), Threonine (T), Cysteine (C); and 8) Cysteine (C), Methionine (M) (see, e.g., Creighton, Proteins (1984)).

The term “nucleic acid” refers to a deoxyribonucleotide or ribonucleotide polymer in either single- or double-stranded form, and unless otherwise limited, encompasses known analogues of natural nucleotides that hybridize to nucleic acids in a manner similar to naturally occurring nucleotides. Unless otherwise indicated, a particular nucleic acid sequence includes the complementary sequence thereof. The terms “nucleic acid”, “nucleic acid sequence”, and “polynucleotide” are used interchangeably herein.

The term “operably linked” refers to functional linkage between a nucleic acid expression control sequence (such as a promoter, signal sequence, or array of transcription factor binding sites) and a second nucleic acid sequence, wherein the expression control sequence affects transcription and/or translation of the nucleic acid corresponding to the second sequence.

The term “recombinant” when used with reference to a cell indicates that the cell replicates a heterologous nucleic acid, or expresses a peptide or protein encoded by a heterologous nucleic acid. Recombinant cells can contain genes that are not found within the native (non-recombinant) form of the cell. Recombinant cells can also contain genes found in the native form of the cell wherein the genes are modified and re-introduced into the cell by artificial means. The term also encompasses cells that contain a nucleic acid endogenous to the cell that has been modified without removing the nucleic acid from the cell; such modifications include those obtained by gene replacement, site-specific mutation, and related techniques.

A “recombinant nucleic acid” refers to a nucleic acid that was artificially constructed (e.g., formed by linking two naturally-occurring or synthetic nucleic acid fragments). This term also applies to nucleic acids that are produced by replication or transcription of a nucleic acid that was artificially constructed. A “recombinant polypeptide” is expressed by transcription of a recombinant nucleic acid (i.e., a nucleic acid that is not native to the cell or that has been modified from its naturally occurring form), followed by translation of the resulting transcript.

A “heterologous polynucleotide” or a “heterologous nucleic acid”, as used herein, is one that originates from a source foreign to the particular host cell, or, if from the same source, is modified from its original form. Thus, a heterologous TRIM62 or p27 gene in a prokaryotic host cell includes a TRIM62 or p27 gene that is endogenous to the particular host cell but has been modified. Modification of the heterologous sequence may occur, e.g., by treating the DNA with a restriction enzyme to generate a DNA fragment that is capable of being operably linked to a promoter. Techniques such as site-directed mutagenesis are also useful for modifying a heterologous sequence.

A “subsequence” refers to a sequence of nucleic acids or amino acids that comprise a part of a longer sequence of nucleic acids or amino acids (e.g., polypeptide) respectively.

A “recombinant expression cassette” or simply an “expression cassette” is a nucleic acid construct, generated recombinantly or synthetically, with nucleic acid elements that are capable of affecting expression of a structural gene in hosts compatible with such sequences. Expression cassettes include at least promoters and optionally, transcription termination signals. Typically, the recombinant expression cassette includes a nucleic acid to be transcribed (e.g., a nucleic acid encoding a desired polypeptide), and a promoter. Additional factors necessary or helpful in effecting expression may also be used as described herein. For example, an expression cassette can also include nucleotide sequences that encode a signal sequence that directs secretion of an expressed protein from the host cell. Transcription termination signals, enhancers, and other nucleic acid sequences that influence gene expression, can also be included in an expression cassette.

The term “isolated” refers to material that is substantially or essentially free from components which interfere with the activity of an enzyme. For cells, saccharides, nucleic acids, and polypeptides of the invention, the term “isolated” refers to material that is substantially or essentially free from components which normally accompany the material as found in its native state. Typically, isolated saccharides, proteins or nucleic acids of the invention are at least about 50%, 55%, 60%, 65%, 70%, 75%, 80% or 85% pure, usually at least about 90%, 91%, 92%, 93%, 94%, 95%, 96%, 97%, 98%, or 99% pure as measured by band intensity on a silver stained gel or other method for determining purity. Purity or homogeneity can be indicated by a number of means well known in the art, such as polyacrylamide gel electrophoresis of a protein or nucleic acid sample, followed by visualization upon staining. For certain purposes high resolution will be needed and HPLC or a similar means for purification utilized. For oligonucleotides, or other galactosylated products, purity can be determined using, e.g., thin layer chromatography, HPLC, or mass spectroscopy.

The terms “identical” or percent “identity,” in the context of two or more nucleic acid or polypeptide sequences, refer to two or more sequences or subsequences that are the same or have a specified percentage of amino acid residues or nucleotides that are the same, when compared and aligned for maximum correspondence, as measured using one of the following sequence comparison algorithms or by visual inspection.

The phrase “substantially identical,” in the context of two nucleic acids or polypeptides, refers to two or more sequences or subsequences that have at least 60%, preferably 80% or 85%, most preferably at least 90%, 91%, 92%, 93%, 94%, 95%, 96%, 97%, 98%, or 99% nucleotide or amino acid residue identity, when compared and aligned for maximum correspondence, as measured using one of the following sequence comparison algorithms or by visual inspection. Preferably, the substantial identity exists over a region of the sequences that is at least about 50 residues in length, more preferably over a region of at least about 100 residues, and most preferably the sequences are substantially identical over at least about 150 residues. In a most preferred embodiment, the sequences are substantially identical over the entire length of the coding regions.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)).

Examples of algorithms that are suitable for determining percent sequence identity and sequence similarity are the BLAST and BLAST 2.0 algorithms, which are described in Altschul et al. (1990) J. Mol. Biol. 215: 403-410 and Altschuel et al. (1977) Nucleic Acids Res. 25: 3389-3402, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions, as described below.

The phrase “hybridizing specifically to”, refers to the binding, duplexing, or hybridizing of a molecule only to a particular nucleotide sequence under stringent conditions when that sequence is present in a complex mixture (e.g., total cellular) DNA or RNA.

The term “stringent conditions” refers to conditions under which a probe will hybridize to its target subsequence, but to no other sequences. Stringent conditions are sequence-dependent and will be different in different circumstances. Longer sequences hybridize specifically at higher temperatures. Generally, stringent conditions are selected to be about 5° C. lower than the thermal melting point (Tm) for the specific sequence at a defined ionic strength and pH. The Tm is the temperature (under defined ionic strength, pH, and nucleic acid concentration) at which 50% of the probes complementary to the target sequence hybridize to the target sequence at equilibrium. (As the target sequences are generally present in excess, at Tm, 50% of the probes are occupied at equilibrium). Typically, stringent conditions will be those in which the salt concentration is less than about 1.0 M Na⁺ ion, typically about 0.01 to 1.0 M Na⁺ ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions can also be achieved with the addition of destabilizing agents such as formamide. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90-95° C. for 30-120 sec, an annealing phase lasting 30-120 sec, and an extension phase of about 72° C. for 1-2 min. Protocols and guidelines for low and high stringency amplification reactions are available, e.g., in Innis, et al. (1990) PCR Protocols: A Guide to Methods and Applications Academic Press, N.Y.

The phrases “specifically binds to” or “specifically immunoreactive with”, when referring to an antibody refers to a binding reaction which is determinative of the presence of the protein or other antigen in the presence of a heterogeneous population of proteins, saccharides, and other biologics. Thus, under designated immunoassay conditions, the specified antibodies bind preferentially to a particular antigen and do not bind in a significant amount to other molecules present in the sample. Specific binding to an antigen under such conditions requires an antibody that is selected for its specificity for a particular antigen. A variety of immunoassay formats can be used to select antibodies specifically immunoreactive with a particular antigen. For example, solid-phase ELISA immunoassays are routinely used to select monoclonal antibodies specifically immunoreactive with an antigen. See Harlow and Lane (1988) Antibodies, A Laboratory Manual, Cold Spring Harbor Publications, New York, for a description of immunoassay formats and conditions that can be used to determine specific immunoreactivity.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. In a preferred embodiment, antibodies that specifically bind to a TRIM62 or p27 protein are produced. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F (ab)′₂, a dimer of Fab which itself is a light chain joined to V_{H}-C_{H}1 by a disulfide bond. The F (ab)′₂ may be reduced under mild conditions to break the disulfide linkage in the hinge region, thereby converting the F (ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments can be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

For preparation of antibodies, e.g., recombinant, monoclonal, or polyclonal antibodies, many technique known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow and Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3^{rd} ed. 1997)). Techniques for the production of single chain antibodies or recombinant antibodies (U.S. Pat. No. 4,946,778, U.S. Pat. No. 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

In one embodiment, the antibody is conjugated to an “effector” moiety. The effector moiety can be any number of molecules, including labeling moieties such as radioactive labels or fluorescent labels for use in diagnostic assays.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies raised to IgE protein, polymorphic variants, alleles, orthologs, and conservatively modified variants, or splice variants, or portions thereof, can be selected to obtain only those polyclonal antibodies that are specifically immunoreactive with IgE proteins and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow and Lane, Antibodies, A Laboratory Manual (1988) for a description of immunoassay formats and conditions that can be used to determine specific immunoreactivity).

An “antigen” is a molecule that is recognized and bound by an antibody, e.g., peptides, carbohydrates, organic molecules, or more complex molecules such as glycolipids and glycoproteins. The part of the antigen that is the target of antibody binding is an antigenic determinant and a small functional group that corresponds to a single antigenic determinant is called a hapten.

A “label” is a composition detectable by spectroscopic, photochemical, biochemical, immunochemical, or chemical means. For example, useful labels include ³²P, ¹²⁵I, fluorescent dyes, electron-dense reagents, enzymes (e.g., as commonly used in an ELISA), biotin, digoxigenin, or haptens and proteins for which antisera or monoclonal antibodies are available (e.g., the polypeptide of SEQ ID NO:2 can be made detectable, e.g., by incorporating a radiolabel into the peptide, and used to detect antibodies specifically reactive with the peptide).

The term “immunoassay” is an assay that uses an antibody to specifically bind an antigen. The immunoassay is characterized by the use of specific binding properties of a particular antibody to isolate, target, and/or quantify the antigen.

The term “carrier molecule” means an immunogenic molecule containing antigenic determinants recognized by T cells. A carrier molecule can be a protein or can be a lipid. A carrier protein is conjugated to a polypeptide to render the polypeptide immunogenic. Carrier proteins include keyhole limpet hemocyanin, horseshoe crab hemocyanin, and bovine serum albumin.

The term “adjuvant” means a substance that nonspecifically enhances the immune response to an antigen. Adjuvants include, for example, Freund's adjuvant, either complete or incomplete; Titermax® gold adjuvant; alum; and bacterial LPS.

The term “contacting” is used herein interchangeably with the following: combined with, added to, mixed with, passed over, incubated with, flowed over, etc.

By “therapeutically effective dose” herein is meant a dose that produces effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); and Pickar, Dosage Calculations (1999)).

As used herein, “cancer” includes solid tumors and hematological malignancies. The former includes cancers such as breast, colon, and ovarian cancers. The latter include hematopoietic malignancies including leukemias, lymphomas and myelomas. This invention provides new effective methods, compositions, and kits for treatment and/or prevention of various types of cancer.

Hematological malignancies, such as leukemias and lymphomas, are conditions characterized by abnormal growth and maturation of hematopoietic cells.

Leukemias are generally neoplastic disorders of hematopoietic stem cells, and include adult and pediatric acute myeloid leukemias (AML), chronic myeloid leukemia (CML), acute lymphocytic leukemia (ALL), chronic lymphocytic leukemia (CLL), hairy cell leukemia and secondary leukemia. Myeloid leukemias are characterized by infiltration of the blood, bone marrow, and other tissues by neoplastic cells of the hematopoietic system. CLL is characterized by the accumulation of mature-appearing lymphocytes in the peripheral blood and is associated with infiltration of bone marrow, the spleen and lymph nodes.

Specific leukemias include acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia.

Lymphomas are generally neoplastic transformations of cells that reside primarily in lymphoid tissue. Among lymphomas, there are two major distinct groups: non-Hodgkin's lymphoma (NHL) and Hodgkin's disease. Lymphomas are tumors of the immune system and generally are present as both T cell- and as B cell-associated disease. Bone marrow, lymph nodes, spleen and circulating cells are all typically involved. Treatment protocols include removal of bone marrow from the patient and purging it of tumor cells, often using antibodies directed against antigens present on the tumor cell type, followed by storage. The patient is then given a toxic dose of radiation or chemotherapy and the purged bone marrow is then reinfused in order to repopulate the patient's hematopoietic system.

Other hematological malignancies include myelodysplastic syndromes (MDS), myeloproliferative syndromes (MPS) and myelomas, such as solitary myeloma and multiple myeloma. Multiple myeloma (also called plasma cell myeloma) involves the skeletal system and is characterized by multiple tumorous masses of neoplastic plasma cells scattered throughout that system. It may also spread to lymph nodes and other sites such as the skin. Solitary myeloma involves solitary lesions that tend to occur in the same locations as multiple myeloma.

Hematological malignancies are generally serious disorders, resulting in a variety of symptoms, including bone marrow failure and organ failure. Treatment for many hematological malignancies, including leukemias and lymphomas, remains difficult, and existing therapies are not universally effective. While treatments involving specific immunotherapy appear to have considerable potential, such treatments have been limited by the small number of known malignancy-associated antigens. Moreover the ability to detect such hematological malignancies in their early stages can be quite difficult depending upon the particular malady. Accordingly, there remains a need in the art for improved methods for treatment of hematological malignancies such as B cell leukemias and lymphomas and multiple myelomas. The present invention fulfills these and other needs in the field.

Other cancers are also of concern, and represent similar difficulties insofar as effective treatment is concerned. Such cancers include those characterized by solid tumors. Examples of other cancers of concern are skin cancers, including melanomas, basal cell carcinomas, and squamous cell carcinomas. Epithelial carcinomas of the head and neck are also encompassed by the present invention. These cancers typically arise from mucosal surfaces of the head and neck and include salivary gland tumors.

The present invention also encompasses cancers of the lung. Lung cancers include squamous or epidermoid carcinoma, small cell carcinoma, adenocarcinoma, and large cell carcinoma. Breast cancer is also included, both invasive breast cancer and non-invasive breast cancer, e.g., ductal carcinoma in situ and lobular neoplasia.

The present invention also encompasses gastrointestinal tract cancers. Gastrointestinal tract cancers include esophageal cancers, gastric adenocarcinoma, primary gastric lymphoma, colorectal cancer, small bowel tumors and cancers of the anus. Pancreatic cancer and cancers that affect the liver are also of concern, including hepatocellular cancer.

The present invention also includes treatment of bladder cancer and renal cell carcinoma.

The present invention also encompasses prostatic carcinoma and testicular cancer.

Gynecologic malignancies are also encompassed by the present invention including ovarian cancer, carcinoma of the fallopian tube, uterine cancer, and cervical cancer.

Treatment of sarcomas of the bone and soft tissue are encompassed by the present invention. Bone sarcomas include osteosarcoma, chondrosarcoma, and Ewing's sarcoma.

The present invention also encompasses malignant tumors of the thyroid, including papillary, follicular, and anaplastic carcinomas.

### III. TRIM62, p27^{Kip1}, and EGFR Family Members

The present invention is based on the discovery that p27^{Kip1} protein levels are regulated by the activity of the TRIM62 protein. Inhibition of TRIM62 activity results in increased expression and activity of the p27^{Kip1} protein. In some embodiments, inhibition of TRIM62 activity results in higher levels of p27^{Kip1} protein the cell nucleus. Some inhibitors of TRIM62 expression and activity are commercially available, e.g., TRIM62 specific siRNA molecules from, e.g., Invitrogen.

Those of skill will recognize that measurement of TRIM62 expression or activity is useful for the methods described herein. TRIM62 expression can be measured at the nucleic acid level, i.e., mRNA or DNA, or at the protein level. Standard molecular techniques can be used to perform such measurements, e.g., Northern blots, Southern blots, Western blots, immunohistochemistry, quantitative PCR techniques, such as real time PCR. Antibodies that specifically bind to the TRIM62 protein are available, from, e.g., AbCam.

TRIM62 activity is measured using, e.g., E3 ligase assays. Such assays are described in e.g., Davydov, et al., Soc. Biomol. Screen. 9:695-703 (2004) and include high throughput assays. TRIM62 activity is also measured using cell based activities, e.g., by determining p27^{Kip1} protein expression levels in a cell. p27^{Kip1} protein levels can be determined using assays for protein detection, e.g., Western blotting, or by using biochemical assays for inhibition of cyclin-CDK activity.

In some embodiments, it is beneficial to determine the level of expression of an EGFR family member by a cancer cell. EGFR family proteins are receptor tyrosine kinase that stimulate cell division when activated. Excessive and inappropriate signaling by EGFR proteins is associated with a wide variety of cancers. EGFR proteins have a cytoplasmic tyrosine kinase domain, a single transmembrane region, and an extracellular region with four sub-domains, L1 (leucine-rich), CR1 (cysteine-rich), L2, and CR2. Examples of members of the EGFR family are ErbB1, also called epidermal growth factor receptor (EGFR); ErbB2, also called HER2 or neu; ErbB3, also called HER3; and ErbB4, also called HER4.

Compounds that inhibit activity of EGFR family members are in use as therapeutics for treatment of cancers that express or overexpress and EGFR family member. Examples are trastuzumab, an antibody that recognizes ErbB2; lapatinib, a small molecule that inhibits ErbB1 and ErbB2; gefitinib and erlotinib are small molecules that inhibit ErbB1; cetuximab and panitumumab are antibodies that recognize and inhibit ErbB1. Other molecules that inhibit members of the EGFR family include, e.g., pertuzumab, and canertinib. Methods to make and use inhibitors of EGFR protein tyrosine kinases are known to those of skill in the art. See e.g., Shawver, et al., Cancer Cell 1:117-123 (2002); de Bono and Rowinsky, Trends Mol. Med. 8:S19-S26 (2002). Any of these agents can be used in combination with a TRMI62 inhibitor to treat a cancer that has increased activity or expression of an EGFR family member.

Examples of cancers that exhibit increased activity or expression of the EGFR protein include head and neck, non-small cell lung cancer (NSCLC), laryengeal, esophageal, gastric, pancreatic, colon, renal cell, bladder, breast, ovarian, cervical, prostate, papillary thyroid cancers, melanoma, and gliomas. See e.g., Shawver, et al., Cancer Cell 1:117-123 (2002). Examples of cancers that exhibit increased activity or expression of the ErbB2 protein include breast cancer, ovarian cancer, prostate cancer, and gastric cancer.

Examples of cancers that exhibit increased activity or expression of the EGFR family members, i.e., EGFR, ErbB2, ErbB3, and ErbB4, include adult and pediatric acute myeloid leukemias (AML); chronic myeloid leukemia (CML; acute lymphocytic leukemia (ALL; chronic lymphocytic leukemia (CLL; hairy cell leukemia; secondary leukemia; acute nonlymphocytic leukemia; chronic lymphocytic leukemia; acute granulocytic leukemia; chronic granulocytic leukemia; acute promyelocytic leukemia; adult T-cell leukemia; aleukemic leukemia; a leukocythemic leukemia; basophylic leukemia; blast cell leukemia; bovine leukemia; chronic myelocytic leukemia; leukemia cutis; embryonal leukemia; eosinophilic leukemia; Gross' leukemia; hairy-cell leukemia; hemoblastic leukemia; hemocytoblastic leukemia; histiocytic leukemia; stem cell leukemia; acute monocytic leukemia; leukopenic leukemia; lymphatic leukemia; lymphoblastic leukemia; lymphocytic leukemia; lymphogenous leukemia; lymphoid leukemia; lymphosarcoma cell leukemia; mast cell leukemia; megakaryocytic leukemia; micromyeloblastic leukemia, monocytic leukemia; myeloblastic leukemia; myelocytic leukemia; myeloid granulocytic leukemia; myelomonocytic leukemia; Naegeli leukemia; plasma cell leukemia; plasmacytic leukemia; promyelocytic leukemia; Rieder cell leukemia; Schilling's leukemia; stem cell leukemia; subleukemic leukemia; undifferentiated cell leukemia; non-Hodgkin's lymphoma (NHL); Hodgkin's disease; myelodysplastic syndromes (MDS); myeloproliferative syndromes (MPS); myelomas, such as solitary myeloma and multiple myeloma; skin cancers, including melanomas, basal cell carcinomas, Kaposi's sarcoma, and squamous cell carcinomas; epithelial carcinomas of the head and neck; lung cancers, including squamous or epidermoid carcinoma, small cell carcinoma, adenocarcinoma, and large cell carcinoma; breast cancer, including invasive breast cancer and non-invasive breast cancer; gastrointestinal tract cancers, including esophageal cancers, gastric adenocarcinoma, primary gastric lymphoma, colorectal cancer, small bowel tumors and cancers of the anus; pancreatic cancer and cancers of the liver, including hepatocellular cancer; bladder cancer; renal cell carcinoma; prostatic carcinoma; testicular cancer; ovarian cancer, carcinoma of the fallopian tube; uterine cancer; cervical cancer; sarcomas of the bone and soft tissue, including osteosarcoma, chondrosarcoma, and Ewing's sarcoma; and malignant tumors of the thyroid, including papillary, follicular, and anaplastic carcinomas.

### IV. RNA Interference

A double stranded RNA molecule to interfere with TRIM82 gene expression and protein production. Cellular uptake of double-stranded RNA is efficient, thereby permitting RNA interference to occur with relatively small amounts of dsRNA.

When double-stranded RNA (dsRNA) is introduced into a cell, it has the ability to silence the expression of a homologous gene within the cell, i.e., “interfere” with gene expression. siRNA provides a substrate for the RNA-induced silencing complex (RISC), and is inactive until incorporated into this macromolecular complex.

In eukaryotes, the current model of the RNA interference mechanism involves both an initiation and an effector step. In the initiation step, a processing enzyme cleaves the introduced dsRNA into small interfering RNAs of 21-23 nucleotides. In the effector step, each siRNA is incorporated into an RNA induced silencing complex (“RISC”), comprising a helicase, an exonucleolytic nuclease, and an endonucleolytic nuclease. The siRNA, now incorporated into the RISC, serves as a guide molecule, directing the RISC to the homologous mRNA transcript for degradation. See, e.g., Hammond, et al., Nature Rev. Gen., 2:110-119 (2001). The RISC complex is led to the intended mRNA by the incorporated siRNA molecule and catalyzes the cleavage of multiple copies of the mRNA. Double stranded small interfering molecules have the advantage of being more stable than single stranded RNA, and being more effective at inhibition at lower concentrations than single stranded RNA. In addition, siRNA does not require the use of viral vectors.

Other double stranded RNA molecules are also included within the scope of the invention. A growing number of RNAs do not function as messenger RNAs, transfer RNAs or ribosomal RNAs. These so-called “non-coding” RNAs describe a wide variety of RNAs of incredibly diverse function, ranging from the purely structural to the purely regulatory (Riddihough, (2002) Science, 296:1259). The non-coding RNA that has generated the most interest, however, is the “small interfering RNA” or “siRNA” associated with the phenomenon of RNA interference (“RNAi”). Other representative non-coding RNAs include small nuclear RNAs, involved in the splicing of pre-mRNAs in eukaryotes (Will et al., (2001) Curr. Opin. Cell Biol., 13:290), small nucleolar RNAs, which direct 2′-O-ribose methylation and pseudouridylation of rRNA and tRNA (Kiss, (2001) EMBO J., 20:3617) and “micro-RNAs” (“miRNAs”), very small RNAs of approximately 22 nucleotides in length which appear to be involved in various aspects of mRNA regulation and degradation. Two miRNAs characterized in some detail are the “small temporal RNAs” (“stRNAs”) lin4 and let7, which control developmental timing in the nematode worm C. elegans and repress the translation of their target genes by binding to the 3′ untranslated regions of their mRNAs (Riddihough, (2002) supra); Ruvkun, (2001) Science 294:797; Grosshans et al., (2002) J. Cell. Biol. 156:17). Also known are the short hairpin RNAs (“shRNAs”), patterned from endogenously encoded triggers of the RNA interference pathway (Paddison et al., (2002) Genes Dev. 16:948-958).

siRNA are introduced into the cell rather than large dsRNA molecules, thus circumventing the initiation step of the mechanism. Although composed of two structural elements that resemble oligonucleotides used in antisense gene inhibition, the siRNA molecule has clear structural distinctions from the former. A siRNA molecule is composed of two complementary strands of RNA that must be hybridized with one another. There must be base-pair overhangs at each end of the molecule. Although the two oligonucleotides used for siRNA are the same length as those used for antisense, they will not be incorporated into the RISC complex unless they form this RNA duplex.

Where the siRNA of the present invention is delivered to a cell for the purposes of inhibiting expression of a target gene within the cell, at least one strand of the small interfering RNA is homologous to a portion of mRNA transcribed from the target gene, e.g., TRIM62. In a preferred embodiment, the siRNA strand is at least 85% homologous to a portion of mRNA transcribed from the target gene. Preferably, the siRNA strand is 90% identical, more preferably is 95% identical, and even more preferably, is 98% and 99% identical to a portion of mRNA transcribed from the target gene, e.g., TRIM62. In the most preferred embodiment, at least one strand of the siRNA is 100% identical to a portion of mRNA transcribed from the target gene, e.g., TRIM62.

In one embodiment, at least one siRNA molecule can be delivered to the cell, for example an siRNA molecule associated with a region of the TIRM621 gene. In another embodiment, a plurality of siRNA molecules can be delivered to the cell, for example, a plurality of siRNA molecules associated with one region of the TIRM62 gene. In another embodiment, the plurality of siRNA molecules can be associated with different regions of the TRIM62 gene. Thus, it will be appreciated that the scope of the invention covers any combination of siRNA molecules that can target and interfere with one or more desired regions of the TIRM62 gene.

The target gene may be an endogenous gene in relation to the cell, as in the case of a regulatory gene or a gene coding for a native protein, or it may be heterologous in relation to the cell, as in the case of a viral or bacterial gene, transposon, or transgene. In either case, uninhibited expression of the target gene may result in a disease or a condition. To inhibit expression of the target gene, the cell is contacted with the siRNA in an amount sufficient to inhibit expression of the target gene, e.g., TRIM62.

The cell receiving the siRNA of the present invention may be isolated, within a tissue, or within an organism. It may be an animal cell, a plant cell, a fungal cell, a protozoan, or a bacterium. An animal cell may be derived from vertebrates or invertebrates, but in a preferred embodiment of the invention, the cell is derived from a mammal, such as a rodent or a primate, and even more preferably, is derived from a human. The cell may be of any type, including neural cells, neuronal cells, epithelial cells, endothelial cells, muscle cells or nerve cells. Representative cell types include, but are not limited to, microglia, myoblasts, fibroblasts, astrocytes, neurons, oligodendrocytes, macrophages, myotubes, lymphocytes, NIH3T3 cells, PC12 cells, and neuroblastoma cells. In a further preferred embodiment the cell is derived from a cancer or tumor. In some embodiments the cancer or tumor cell exhibits over expression of a member of the epidermal growth factor family of proteins. In one embodiment, the cancer or tumor cell has increased activity of the ErbB2 protein, e.g., increased activity caused by an amplification of the ErbB2 gene. Delivery may be accomplished either in vitro or in vivo by standard techniques.

The siRNA can be obtained by chemical synthesis or by DNA-vector based RNA interference technology. Custom siRNAs can be generated on order from Dharmacon Research, Inc., Lafayette, Colo. Other sources for custom siRNA preparation include Xeragon Oligonucleotides, Huntsville, Ala. and Ambion of Austin, Tex. Alternatively, siRNAs can be chemically synthesized using ribonucleoside phosphoramidites and a DNA/RNA synthesizer. In the present invention, the siRNA molecules were chemically synthesized using the Invitrogen commercially available technique with ribonucleoside phosphoramidites and a DNA/RNA synthesizer.

Using DNA vector based siRNA technology, a small DNA insert (about 70 bp) encoding a short hairpin RNA targeting the gene of interest is cloned into a commercially available vector. The insert-containing vector can be transfected into the cell, and it expresses the short hairpin RNA. The hairpin RNA is rapidly processed by the cellular machinery into 19-22 nt double stranded RNA (siRNA). The following is a list of commercially available GenScript siRNA expression vectors: U6 like promoter: pRNA-U6.1/Neo, pRNA-U6.1/Hygro, pRNA-U6.1/Zeo, pRNAT-U6.1/Neo (with GFP marker), pRNAT-U6.1/Hygro (with GFP marker). H1 like promoter: pRNA-H1.1/Neo, pRNA-H1.1/Hygro, pRNA-H1.1/Zeo, pRNAT-H1.1/Neo (with GFP marker), pRNAT-H1.1/Hygro (with GFP marker).

To improve hybridization, locked bases, which differ from native RNA bases in that they contain a 2′-O, 4′-C methylene bridge, can be used. By chemically modifying the siRNA, enhanced hybridization and improved biostability, can be achieved. The siRNA can be chemically modified at either or both the 5′ and 3′ end bases to increase stability, hybridization, and cellular uptake. The molecules can be modified using the locked base technology described by Proligo in U.S. Pat. No. 6,794,499 and U.S. Pat. No. 6,670,461, incorporated herein by reference.

The siRNA can be chemically modified, for example, by N-type modification to produce a linked nucleic acid (LNA). LNA is a synthetic nucleic acid analogue, incorporating “internally bridged” nucleoside analogues. Synthesis of LNA, and properties thereof, have been described by a number of authors: Nielsen et al., (1997) J. Chem. Soc. Perkin Trans. 1:3423); Koshkin et al., (1998) Tetrahedron Lett. 39:4381; Singh and Wengel (1998) Chem. Commun. 1247; and Singh et al., (1998) Chem. Commun. 455. LNA exhibits greater thermal stability when paired with DNA, than do conventional DNA/DNA heteroduplexes.

A sugar engineered into an N-type (RNA-like) pucker usually conveys an increase in helical thermostability when hybridized with complementary RNA (Freier et al., (1997) Nucleic Acids Res. 25:4429-4443). Prominent examples of such N-type nucleic acid analogues are 2′-O-alkylated RNA (Manoharan (1999) Biochim. Biophys. Acta 1489:117-130), 2′F-RNA (Kawasaki et al. (1993) J. Med. Chem. 36:831-841), phosphoramidates (Gryaznov (1999) Biochim. Biophys. Acta 1489, 131-140), HNA (Hendrix et al. (1997) Chem. Eur. J. 3:1513-1520), and LNA (Koshkin, et al. (1998) Tetrahedron 54:3607-3630; Obika, et al. (1998) Tetrahedron Lett. 39:5401-5404; Wengel, (1999) Acc. Chem. Res. 32:301-310; and Petersen (2003) Trends Biotechnol. 21:74-81).

In LNA, the furanose conformation is chemically locked in an N-type (C3′-endo) conformation by the introduction of a 2′-O,4′-C methylene linkage. LNAs have shown high thermal affinities when hybridized with either DNA (Tm=1-8.degree. C. per modification) (Koshkin et al. (1998) Tetrahedron 54:3607-3630; Obika, et al. (1998) Tetrahedron Lett. 39:5401-5404; Wengel. (1999) Acc. Chem. Res. 32:301-310; Petersen, et al. (2003) Trends Biotechnol. 21:74-81; Kvaern, et al. (2000) J. Org. Chem. 65:5167-5176; and Braasch, et al. (2001) Chem. Biol. 8:1-7), RNA (Tm=2-10.degree. C. per modification) (Braasch, (2001) Chem. Biol. 8:1-7; Bondensgaard, et al. (2000) Chem. Eur. J. 6:2687-2695; and Kurreck et al. (2002) Nucleic Acids Res. 30:1911-1918) or LNA (Tm>5.degree. C. per modification) (Koshkin, (1998) J. Am. Chem. Soc. 120:13252-13253).

In one embodiment, the invention pertains to eliminating the TRIM62 protein by causing the degradation of the mRNA encoding TRIM62 protein using dsRNA, interference, specifically with siRNA molecules.

RNA interference with siRNA produces a measurable reduction of expression of a target gene or a target protein, e.g., a TRIM72 protein. Preferably a reduction in expression is at least about 10%. More preferably the reduction of expression is about 20%, 30%, 40%, 50%, 60%, 80%, 90% and even more preferably, about 100%.

Previous methods of delivering double stranded RNA primarily involve transfection (for general transfection protocols, see Elbashir et al., (2001) Nature 411:494-498; Elbashir et al., (2001) Genes Dev. 15:188-200). The efficiency of transfection depends on cell type, passage number and the confluency of the cells. The time and the manner of formation of dsRNA are also critical. One example of transfection of siRNA molecules includes using U6 and CMV promoters in any suitable transfection vector.

Another method of delivering double stranded molecules to a cell involves using cell-penetration enhancing peptides conjugated to the double stranded molecules. The membrane shuttling proteins such as the Drosophila homeobox protein Antennapedia, the HIV-1 transcriptional factor TAT and VP22 from HSV-1 can be conjugated to the siRNA molecule to increase its cellular uptake and thus efficacy.

Other techniques for dsRNA uptake include electroporation, injection, liposome-facilitated transport, and microinjection. Although direct microinjection of dsRNA into cells is generally considered to be the most effective means known for inducing RNA interference, the characteristics of this technique severely limit its practical utility. In particular, direct microinjection can only be performed in vitro, which limits its application to gene therapy. Furthermore, only one cell at a time can be microinjected, which limits the technique's efficiency. As a means of introducing dsRNA into cells, electroporation is also relatively impractical because it is not possible in vivo. Finally, while dsRNA can be introduced into cells using liposome-facilitated transportation or passive uptake. The siRNA sequences can be assessed for their ability to inhibit gene expression in cultured cells in the absence of a transfection reagent. In a preferred embodiment, the siRNA is delivered intraspinally without a gene therapy vector. Delivery of siRNA molecules can also be accomplished by passive cellular uptake in vivo (see U.S. Patent Appl. 2004/0248174).

It is also possible to introduce dsRNA indirectly into cells, by transforming the cells with expression vectors containing DNA coding for dsRNA (See, e.g., U.S. Pat. No. 6,278,039, U.S. Patent Appl. 2002/0006664, WO 99/32619, WO 01/29058, WO 01/68836, and WO 01/96584). Cells transformed with the dsRNA-encoding expression vector will then produce dsRNA in vivo.

Where delivery is made in vivo to a living organism, administration may be by any procedure known in the art, including but not limited to, oral, parenteral, intraspinal, intracisternal, subdural, rectal, intradermal, transdermal, intramuscular, or topical administration. To facilitate delivery, the dsRNA may be formulated in various compositions with a pharmaceutically acceptable carrier, excipient or diluent. “Pharmaceutically acceptable” means the carrier, excipient or diluent of choice does not adversely affect the biological activity of the dsRNA, or the recipient of the composition.

Suitable pharmaceutical carriers, excipients and/or diluents include, but are not limited to, lactose, sucrose, starch powder, talc powder, cellulose esters of alkonoic acids, magnesium stearate, magnesium oxide, crystalline cellulose, methyl cellulose, carboxymethyl cellulose, gelatin, glycerin, sodium alginate, gum arabic, acacia gum, sodium and calcium salts of phosphoric and sulfuric acids, polyvinylpyrrolidone and/or polyvinyl alcohol, saline, and water.

For oral administration, the composition may be presented as capsules or tablets, powders, granules or a suspension. The composition may be further presented in convenient unit dosage form, and may be prepared using a controlled-release formulation, buffering agents and/or enteric coatings.

For parenteral administration (i.e., subcutaneous, intravenous, or intramuscular administration), the dsRNA may be dissolved or suspended in a sterile aqueous or non-aqueous isotonic solution, containing one or more of the carriers, excipients or diluents noted above. Such formulations may be prepared by dissolving a composition containing the dsRNA in sterile water containing physiologically compatible substances such as sodium chloride, glycine, and the like, and having a buffered pH compatible with physiological conditions to produce an aqueous solution. Alternatively, a composition containing the dsRNA may be dissolved in non-aqueous isotonic solutions of polyethylene glycol, propylene glycol, ethanol, corn oil, cottonseed oil, peanut oil, etc.

The dsRNA may be administered by formulation with any suitable carrier that is solid at room temperature but dissolves at body temperature. Such carriers include cocoa butter, synthetic mono-, di-, or tri-glycerides, fatty acids, polyethylene glycols, glycerinated gelatin, hydrogenated vegetable oils, and the like.

Intradermal administration of the dsRNA, i.e., administration via injectable preparation, can be accomplished by suspending or dissolving the dsRNA in a non-toxic parenterally acceptable diluent or solvent, e.g., as a solution in 1,3-butanediol, water, Ringer's solution, and isotonic sodium chloride solution. Occasionally, sterile fixed oils or fatty acids are employed as a solvent or suspending medium.

For transdermal or topical administration, the dsRNA can be combined with compounds that act to increase the permeability of the skin and allow passage of the dsRNA into the bloodstream. Such enhancers include propylene glycol, polyethylene glycol, isopropanol, ethanol, oleic acid, N-methylpyrrolidone, and the like. Delivery of such compositions may be via transdermal patch or iontophoresis device.

Specific formulations of compounds for therapeutic treatment are discussed in Hoover, J. E., Remington's Pharmaceutical Sciences (Easton, Pa.: Mack Publishing Co., 1975) and Liberman, H. A., and Lachman, L., Eds., Pharmaceutical Dosage Forms (New York, N.Y.: Marcel Decker Publishers, 1980).

The quantity of dsRNA administered to tissue or to a subject should be an amount that is effective to inhibit expression of the target gene within the tissue or subject, and are readily determined by the practitioner skilled in the art. Specific dosage will depend further upon the dsRNA, e.g., siRNA used, the target gene to be inhibited and the cell type having target gene expression. Quantities will be adjusted for the body weight of the subject and the particular disease or condition being targeted.

### V. Detection of Gene Expression

Those of skill will recognize methods to determine expression levels of gene products, e.g., TRIM62 nucleic acids and proteins, p27^{Kip1} nucleic acids and proteins, and various proteins and nucleic aicds from the EGFR family. Gene expression can be determined by measuring mRNA levels or protein levels of interest. In some embodiments levels of cytokines will be determined.

Amplification-based assays can be used to detect the presence of a nucleic acid of interest in a sample. In such amplification-based assays, the nucleic acid sequences act as a template in an amplification reaction (e.g. Polymerase Chain Reaction (PCR). In a quantitative amplification, the amount of amplification product will be proportional to the amount of template in the original sample. Comparison to appropriate (e.g. healthy tissue) controls provides a measure of the copy number.

Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. Detailed protocols for quantitative PCR are provided in Innis et al. (1990) PCR Protocols, A Guide to Methods and Applications, Academic Press, Inc. N.Y.). The known nucleic acid sequence for the genes is sufficient to enable one of skill to routinely select primers to amplify any portion of the gene.

Real time PCR is another amplification technique that can be used to determine gene copy levels or levels of mRNA expression. (See, e.g., Gibson et al., Genome Research 6:995-1001, 1996; Heid et al., Genome Research 6:986-994, 1996). Real-time PCR is a technique that evaluates the level of PCR product accumulation during amplification. This technique permits quantitative evaluation of mRNA levels in multiple samples. For gene copy levels, total genomic DNA is isolated from a sample. For mRNA levels, mRNA is extracted from tumor and normal tissue and cDNA is prepared using standard techniques. Real-time PCR can be performed, for example, using a Perkin Elmer/Applied Biosystems (Foster City, Calif.) 7700 Prism instrument. Matching primers and fluorescent probes can be designed for genes of interest using, for example, the primer express program provided by Perkin Elmer/Applied Biosystems (Foster City, Calif.). Optimal concentrations of primers and probes can be initially determined by those of ordinary skill in the art, and control (for example, β-actin) primers and probes may be obtained commercially from, for example, Perkin Elmer/Applied Biosystems (Foster City, Calif.). To quantitate the amount of the specific nucleic acid of interest in a sample, a standard curve is generated using a control. Standard curves can be generated using the Ct values determined in the real-time PCR, which are related to the initial concentration of the nucleic acid of interest used in the assay. Standard dilutions ranging from 10-10⁶ copies of the gene of interest are generally sufficient. In addition, a standard curve is generated for the control sequence. This permits standardization of initial content of the nucleic acid of interest in a tissue sample to the amount of control for comparison purposes.

Other suitable amplification methods include, but are not limited to ligase chain reaction (LCR) (see Wu and Wallace (1989) Genomics 4: 560, Landegren et al. (1988) Science 241: 1077, and Barringer et al. (1990) Gene 89: 117), transcription amplification (Kwoh et al. (1989) Proc. Natl. Acad. Sci. USA 86: 1173), self-sustained sequence replication (Guatelli et al. (1990) Proc. Natl. Acad. Sci. USA 87: 1874), dot PCR, and linker adapter PCR.

Methods of detecting and/or quantifying the gene transcript (mRNA or cDNA) using nucleic acid hybridization techniques are known to those of skill in the art (see Sambrook et al., supra). For example, one method for evaluating the presence, absence, or quantity of mRNA involves a Northern blot transfer.

The probes can be full length or less than the full length of the nucleic acid sequence encoding the protein. Shorter probes are empirically tested for specificity. Preferably nucleic acid probes are 20 bases or longer in length. (See Sambrook et al. for methods of selecting nucleic acid probe sequences for use in nucleic acid hybridization.) Visualization of the hybridized portions allows the qualitative determination of the presence or absence of mRNA.

In another preferred embodiment, a transcript (e.g., mRNA) can be measured using amplification (e.g. PCR) based methods as described above. In a preferred embodiment, transcript level is assessed by using reverse transcription PCR (RT-PCR). In another preferred embodiment, transcript level is assessed by using real-time PCR.

The expression level of a gene of interest can also be detected and/or quantified by detecting or quantifying the encoded polypeptide. The polypeptide can be detected and quantified by any of a number of means well known to those of skill in the art. These may include analytic biochemical methods such as electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunoelectrophoresis, radioimmunoassay (RIA), enzyme-linked immunosorbent assays (ELISAs), immunofluorescent assays, Western blotting, and the like. Immunohistochemical methods can also be used to detect interferon protein. With immunohistochemical staining techniques, a cell sample is prepared, typically by dehydration and fixation, followed by reaction with labeled antibodies specific for the gene product coupled, where the labels are usually visually detectable, such as enzymatic labels, fluorescent labels, luminescent labels, and the like. A particularly sensitive staining technique suitable for use in the present invention is described by Hsu et al. (1980) Am. J. Clin. Path. 75:734-738. The isolated proteins can also be sequenced according to standard techniques to identify polymorphisms.

A polypeptide is detected and/or quantified using any of a number of well recognized immunological binding assays (see, e.g., U.S. Pat. Nos. 4,366,241; 4,376,110; 4,517,288; and 4,837,168). For a review of the general immunoassays, see also Asai (1993) Methods in Cell Biology Volume 37: Antibodies in Cell Biology, Academic Press, Inc. New York; Stites & Ten (1991) Basic and Clinical Immunology 7th Edition.

Immunological binding assays (or immunoassays) typically utilize a “capture agent” to specifically bind to and often immobilize the analyte (polypeptide or subsequence). The capture agent is a moiety that specifically binds to the analyte. In a preferred embodiment, the capture agent is an antibody that specifically binds a polypeptide. The antibody (anti-peptide) may be produced by any of a number of means well known to those of skill in the art.

Immunoassays also often utilize a labeling agent to specifically bind to and label the binding complex formed by the capture agent and the analyte. The labeling agent can itself be one of the moieties comprising the antibody/analyte complex. Thus, the labeling agent can be a labeled polypeptide or a labeled anti-antibody. Alternatively, the labeling agent can be a third moiety, such as another antibody, that specifically binds to the antibody/polypeptide complex.

In one preferred embodiment, the labeling agent is a second human antibody bearing a label. Alternatively, the second antibody can lack a label, but it may, in turn, be bound by a labeled third antibody specific to antibodies of the species from which the second antibody is derived. The second can be modified with a detectable moiety, e.g., as biotin, to which a third labeled molecule can specifically bind, such as enzyme-labeled streptavidin. In some embodiments, Western blot analysis is used to detected and or quantify interferon protein.

Other proteins capable of specifically binding immunoglobulin constant regions, such as protein A or protein G can also be used as the label agent. These proteins are normal constituents of the cell walls of streptococcal bacteria. They exhibit a strong non-immunogenic reactivity with immunoglobulin constant regions from a variety of species (see, generally Kronval, et al. (1973) J. Immunol., 111:1401-1406, and Akerstrom (1985) J. Immunol., 135:2589-2542).

Proteins can be detected and/or quantified in cells using immunocytochemical or immunohistochemical methods. IHC (immunohistochemistry) can be performed on paraffin-embedded tumor blocks using a protein-specific antibody, e.g., TRIM62 or p27, and the like. IHC is the method of colorimetric or fluorescent detection of archival samples, usually paraffin-embedded, using an antibody that is placed directly on slides cut from the paraffin block. To detect and/or quantify a protein in, for example tissue culture cells or cells from a subject that are not embedded in paraffin (for example, hematopoetic cells) ICC (immunocytochemistry) can be used. ICC is like IHC but uses fresh, non-paraffin embedded cells plated onto slides and then fixed and stained.

Either polyclonal or monoclonal antibodies may be used in the immunoassays of the invention described herein. Polyclonal antibodies are preferably raised by multiple injections (e.g. subcutaneous or intramuscular injections) of substantially pure polypeptides or antigenic polypeptides into a suitable non-human mammal. The antigenicity of peptides can be determined by conventional techniques to determine the magnitude of the antibody response of an animal that has been immunized with the peptide. Generally, the peptides that are used to raise the anti-peptide antibodies should generally be those which induce production of high titers of antibody with relatively high affinity for the polypeptide.

Preferably, the antibodies produced will be monoclonal antibodies (“mAb's”). For preparation of monoclonal antibodies, immunization of a mouse or rat is preferred. Polyclonal antibodies can also be used.

It is also possible to evaluate an mAb to determine whether it has the same specificity as a mAb of the invention without undue experimentation by determining whether the mAb being tested prevents a mAb of the invention from binding to the subject gene product isolated as described above. If the mAb being tested competes with the mAb of the invention, as shown by a decrease in binding by the mAb of the invention, then it is likely that the two monoclonal antibodies bind to the same or a closely related epitope. Still another way to determine whether a mAb has the specificity of a mAb of the invention is to preincubate the mAb of the invention with an antigen with which it is normally reactive, and determine if the mAb being tested is inhibited in its ability to bind the antigen. If the mAb being tested is inhibited then, in all likelihood, it has the same, or a closely related, epitopic specificity as the mAb of the invention.

### VI. Assays for Modulators of TRIM62 Activity

A. Assays

Modulation of TRIM62 activity disclosed herein, and corresponding modulation of cellular, e.g., tumor cell, proliferation, can be assessed using a variety of in vitro and in vivo assays, including cell-based models. Such assays can be used to test for inhibitors and activators of a TRIM62 protein disclosed herein, and, consequently, inhibitors and activators of cellular proliferation. Such modulators are useful for treating disorders related to pathological cell proliferation. Modulators are tested using either recombinant or naturally occurring protein, preferably human protein.

Measurement of cellular proliferation modulation by TIRM62 activity disclosed herein or a cell expressing such a protein, either recombinant or naturally occurring, can be performed using a variety of assays, in vitro, in vivo, and ex vivo, as described herein. A suitable physical, chemical or phenotypic change that affects activity, e.g., enzymatic activity, cell proliferation, or ligand binding can be used to assess the influence of a test compound on the polypeptide of this invention. When the effects are determined using intact cells or animals, one can also measure a variety of effects, such as, ligand binding, transcriptional changes to both known and uncharacterized genetic markers (e.g., northern blots), changes in protein levels, e.g., p27 levels, changes in subcellular localization of a protein, e.g., p27, changes in cell metabolism, changes related to cellular proliferation, cell surface marker expression, DNA synthesis, marker and dye dilution assays (e.g., GFP and cell tracker assays), contact inhibition, tumor growth in nude mice, etc.

In Vitro Assays

Assays to identify compounds with modulating activity can be performed in vitro. Such assays can used full length TRIM62 protein or a variant thereof, or a fragment, such as an enzymatic domain. Purified recombinant or naturally occurring TRIM62 protein can be used in the in vitro methods of the invention. In addition to purified protein, the recombinant or naturally occurring protein can be part of a cellular lysate or a cell membrane. As described below, the binding assay can be either solid state or soluble. Preferably, the TRIM62 protein or membrane is bound to a solid support, either covalently or non-covalently. Often, the in vitro assays of the invention are ligand binding or ligand affinity assays, either non-competitive or competitive. Other in vitro assays include measuring changes in spectroscopic (e.g., fluorescence, absorbance, refractive index), hydrodynamic (e.g., shape), chromatographic, or solubility properties for the protein.

In one embodiment, a high throughput binding assay is performed in which the TRIM62 protein or a fragment thereof is contacted with a potential modulator and incubated for a suitable amount of time. In one embodiment, the potential modulator is bound to a solid support, and the protein is added. In another embodiment, the protein is bound to a solid support. A wide variety of modulators can be used, as described below, including small organic molecules, peptides, antibodies, and ligand analogs. A wide variety of assays can be used to identify modulator binding, including labeled protein-protein binding assays, electrophoretic mobility shifts, immunoassays, enzymatic assays, and the like. In some cases, the binding of the candidate modulator is determined through the use of competitive binding assays, where interference with binding of a known ligand is measured in the presence of a potential modulator. Either the modulator or the known ligand is bound first, and then the competitor is added. After the protein is washed, interference with binding, either of the potential modulator or of the known ligand, is determined. Often, either the potential modulator or the known ligand is labeled.

Cell-Based In Vivo Assays

In another embodiment, TRIM62 protein is expressed in a cell, and functional changes are assayed to identify modulators of cellular proliferation, e.g., tumor cell proliferation. Cells expressing the proteins of interest can also be used in binding assays and enzymatic assays. Any suitable functional effect can be measured, as described herein. For example, cellular morphology (e.g., cell volume, nuclear volume, cell perimeter, and nuclear perimeter), ligand binding, p27 expression levels or subcellular localization, cyclin-dependent kinase activity, apoptosis, cell surface marker expression, cellular proliferation, DNA synthesis assays (e.g., ³H-thymidine and fluorescent DNA-binding dyes such as BrdU or Hoescht dye with FACS analysis), are all suitable assays to identify potential modulators using a cell based system. Suitable cells for such cell based assays include both primary cancer or tumor cells and cell lines, as described herein.

Cellular TRIM62 polypeptide levels can be determined by measuring the level of protein or mRNA. The level of protein is measured using immunoassays such as Western blotting, ELISA and the like with an antibody that selectively binds to the polypeptide or a fragment thereof. For measurement of mRNA, amplification, e.g., using PCR, LCR, or hybridization assays, e.g., northern hybridization, RNAse protection, dot blotting, are preferred. The level of protein or mRNA is detected using directly or indirectly labeled detection agents, e.g., fluorescently or radioactively labeled nucleic acids, radioactively or enzymatically labeled antibodies, and the like, as described herein.

Alternatively, protein expression can be measured using a reporter gene system. Such a system can be devised using a protein promoter operably linked to a reporter gene such as chloramphenicol acetyltransferase, firefly luciferase, bacterial luciferase, β-galactosidase and alkaline phosphatase. Furthermore, the protein of interest can be used as an indirect reporter via attachment to a second reporter such as red or green fluorescent protein (see, e.g., Mistili & Spector, Nature Biotechnology 15:961-964 (1997)). The reporter construct is typically transfected into a cell. After treatment with a potential modulator, the amount of reporter gene transcription, translation, or activity is measured according to standard techniques known to those of skill in the art.

Animal Models

Animal models of cellular proliferation also find use in screening for modulators of cellular proliferation. Similarly, transgenic animal technology including gene knockout technology, for example as a result of homologous recombination with an appropriate gene targeting vector, or gene overexpression, will result in the absence or increased expression of the protein. The same technology can also be applied to make knock-out cells. When desired, tissue-specific expression or knockout of the protein may be necessary. Transgenic animals generated by such methods find use as animal models of cellular proliferation and are additionally useful in screening for modulators of cellular proliferation.

Knock-out cells and transgenic mice can be made by insertion of a marker gene or other heterologous gene into an endogenous gene site in the mouse genome via homologous recombination. Such mice can also be made by substituting an endogenous gene with a mutated version of the gene, or by mutating an endogenous gene, e.g., by exposure to carcinogens.

A DNA construct is introduced into the nuclei of embryonic stem cells. Cells containing the newly engineered genetic lesion are injected into a host mouse embryo, which is re-implanted into a recipient female. Some of these embryos develop into chimeric mice that possess germ cells partially derived from the mutant cell line. Therefore, by breeding the chimeric mice it is possible to obtain a new line of mice containing the introduced genetic lesion (see, e.g., Capecchi et al., Science 244:1288 (1989)). Chimeric targeted mice can be derived according to Hogan et al., Manipulating the Mouse Embryo: A Laboratory Manual, Cold Spring Harbor Laboratory (1988) and Teratocarcinomas and Embryonic Stem Cells: A Practical Approach, Robertson, ed., IRL Press, Washington, D.C., (1987).

Exemplary Assays

Soft Agar Growth or Colony Formation in Suspension

Normal cells require a solid substrate to attach and grow. When the cells are transformed, they lose this phenotype and grow detached from the substrate. For example, transformed cells can grow in stirred suspension culture or suspended in semi-solid media, such as semi-solid or soft agar. The transformed cells, when transfected with tumor suppressor genes, regenerate normal phenotype and require a solid substrate to attach and grow.

Soft agar growth or colony formation in suspension assays can be used to identify modulators of TRIM62 activity. Typically, transformed host cells (e.g., cells that grow on soft agar) are used in this assay. For example, A549 cell lines can be used. Techniques for soft agar growth or colony formation in suspension assays are described in Freshney, Culture of Animal Cells a Manual of Basic Technique, 3^{rd} ed., Wiley-Liss, New York (1994), herein incorporated by reference. See also, the methods section of Garkavtsev et al. (1996), supra, herein incorporated by reference.

Contact Inhibition and Density Limitation of Growth

Normal cells typically grow in a flat and organized pattern in a petri dish until they touch other cells. When the cells touch one another, they are contact inhibited and stop growing. When cells are transformed, however, the cells are not contact inhibited and continue to grow to high densities in disorganized foci. Thus, the transformed cells grow to a higher saturation density than normal cells. This can be detected morphologically by the formation of a disoriented monolayer of cells or rounded cells in foci within the regular pattern of normal surrounding cells. Alternatively, labeling index with [³H]-thymidine at saturation density can be used to measure density limitation of growth. See Freshney (1994), supra. The transformed cells, when contacted with cellular proliferation modulators, regenerate a normal phenotype and become contact inhibited and would grow to a lower density.

Contact inhibition and density limitation of growth assays can be used to identify modulators which are capable of inhibiting abnormal proliferation and transformation in host cells. Typically, transformed host cells (e.g., cells that are not contact inhibited) are used in this assay. For example, A549 cell lines can be used. In this assay, labeling index with [³H]-thymidine at saturation density is a preferred method of measuring density limitation of growth. Transformed host cells are contacted with a potential modulator and are grown for 24 hours at saturation density in non-limiting medium conditions. The percentage of cells labeling with [³H]-thymidine is determined autoradiographically. See, Freshney (1994), supra. The host cells contacted with a modulator would give arise to a lower labeling index compared to control (e.g., transformed host cells transfected with a vector lacking an insert).

Growth Factor or Serum Dependence

Growth factor or serum dependence can be used as an assay to identify modulators. Transformed cells have a lower serum dependence than their normal counterparts (see, e.g., Temin, J. Natl. Cancer Insti. 37:167-175 (1966); Eagle et al., J. Exp. Med. 131:836-879 (1970)); Freshney, supra. This is in part due to release of various growth factors by the transformed cells. When transformed cells are contacted with a modulator, the cells would reacquire serum dependence and would release growth factors at a lower level.

Tumor Specific Markers Levels

Tumor cells release an increased amount of certain factors (hereinafter “tumor specific markers”) than their normal counterparts. For example, plasminogen activator (PA) is released from human glioma at a higher level than from normal brain cells (see, e.g., Gullino, Angiogenesis, tumor vascularization, and potential interference with tumor growth. In Mihich (ed.): “Biological Responses in Cancer.” New York, Academic Press, pp. 178-184 (1985)). Similarly, tumor angiogenesis factor (TAF) is released at a higher level in tumor cells than their normal counterparts. See, e.g., Folkman, Angiogenesis and cancer, Sem Cancer Biol. (1992)).

Tumor specific markers can be assayed to identify modulators which decrease the level of release of these markers from host cells. Typically, transformed or tumorigenic host cells are used. Various techniques which measure the release of these factors are described in Freshney (1994), supra. Also, see, Unkless et al., J. Biol. Chem. 249:4295-4305 (1974); Strickland & Beers, J. Biol. Chem. 251:5694-5702 (1976); Whur et al., Br. J. Cancer 42:305-312 (1980); Gulino, Angiogenesis, tumor vascularization, and potential interference with tumor growth. In Mihich, E. (ed): “Biological Responses in Cancer.” New York, Plenum (1985); Freshney Anticancer Res. 5:111-130 (1985).

Invasiveness into an Extracellular Matrix Constituent

The degree of invasiveness into Matrigel™ or some other extracellular matrix constituent can be used as an assay to identify modulators which are capable of inhibiting abnormal cell proliferation and tumor growth. Tumor cells exhibit a good correlation between malignancy and invasiveness of cells into Matrigel™ or some other extracellular matrix constituent. In this assay, tumorigenic cells are typically used as host cells. Therefore, modulators can be identified by measuring changes in the level of invasiveness between the host cells before and after the introduction of potential modulators. If a compound modulates a TRIM62 activity disclosed herein, its expression in tumorigenic host cells would affect invasiveness.

Techniques described in Freshney (1994), supra, can be used. Briefly, the level of invasion of host cells can be measured by using filters coated with Matrigel™ or some other extracellular matrix constituent. Penetration into the gel, or through to the distal side of the filter, is rated as invasiveness, and rated histologically by number of cells and distance moved, or by prelabeling the cells with ¹²⁵I and counting the radioactivity on the distal side of the filter or bottom of the dish. See, e.g., Freshney (1984), supra.

G₀/G₁ Cell Cycle Arrest Analysis

G₀/G₁ cell cycle arrest can be used as an assay to identify modulators. In this assay, cell lines, such as RKO or HCT116, can be used to screen modulators. The cells can be co-transfected with a construct comprising a marker gene, such as a gene that encodes green fluorescent protein, or a cell tracker dye. Methods known in the art can be used to measure the degree of G₁ cell cycle arrest. For example, a propidium iodide signal can be used as a measure for DNA content to determine cell cycle profiles on a flow cytometer. The percent of the cells in each cell cycle can be calculated. Cells contacted with a modulator would exhibit, e.g., a higher number of cells that are arrested in G₀/G₁ phase compared to control.

Tumor Growth In Vivo

Effects of modulators on cell growth can be tested in transgenic or immune-suppressed mice. Knock-out transgenic mice can be made, in which the endogenous gene is disrupted. Such knock-out mice can be used to study effects of a TRIM62 modulator disclosed herein, e.g., as a cancer model, as a means of assaying in vivo for compounds that modulate a TRIM62 activity disclosed herein, and to test the effects of restoring a wild-type TRIM62 activity to a knock-out mice.

Knock-out cells and transgenic mice can be made by insertion of a marker gene or other heterologous gene into the endogenous gene site in the mouse genome via homologous recombination. Such mice can also be made by substituting the endogenous gene with a mutated version of the gene, or by mutating the endogenous gene, e.g., by exposure to carcinogens.

A DNA construct is introduced into the nuclei of embryonic stem cells. Cells containing the newly engineered genetic lesion are injected into a host mouse embryo, which is re-implanted into a recipient female. Some of these embryos develop into chimeric mice that possess germ cells partially derived from the mutant cell line. Therefore, by breeding the chimeric mice it is possible to obtain a new line of mice containing the introduced genetic lesion (see, e.g., Capecchi et al., Science 244:1288 (1989)). Chimeric targeted mice can be derived according to Hogan et al., Manipulating the Mouse Embryo: A Laboratory Manual, Cold Spring Harbor Laboratory (1988) and Teratocarcinomas and Embryonic Stem Cells: A Practical Approach, Robertson, ed., IRL Press, Washington, D.C., (1987). These knock-out mice can be used as hosts to test the effects of various modulators on cell growth.

Alternatively, various immune-suppressed or immune-deficient host animals can be used. For example, genetically athymic “nude” mouse (see, e.g., Giovanella et al., J. Natl. Cancer Inst. 52:921 (1974)), a SCID mouse, a thymectomized mouse, or an irradiated mouse (see, e.g., Bradley et al., Br. J. Cancer 38:263 (1978); Selby et al., Br. J. Cancer 41:52 (1980)) can be used as a host. Transplantable tumor cells (typically about 10⁶ cells) injected into isogenic hosts will produce invasive tumors in a high proportions of cases, while normal cells of similar origin will not. Hosts are treated with modulators, e.g., by injection. After a suitable length of time, preferably 4-8 weeks, tumor growth is measured (e.g., by volume or by its two largest dimensions) and compared to the control. Tumors that have statistically significant reduction (using, e.g., Student's T test) are said to have inhibited growth. Using reduction of tumor size as an assay, modulators which are capable, e.g., of inhibiting abnormal cell proliferation can be identified.

B. Modulators

The compounds tested as modulators of TRIM62 activity can be any small organic molecule, or a biological entity, such as a protein, e.g., an antibody or peptide, a sugar, a nucleic acid, e.g., an siRNA molecule, an antisense oligonucleotide or a ribozyme, or a lipid. Alternatively, modulators can be genetically altered versions of the protein. Typically, test compounds will be a dsRNA molecule, an siRNA molecule, small organic molecules, peptides, lipids, and lipid analogs.

Essentially any chemical compound can be used as a potential modulator or ligand in the assays of the invention, although most often compounds can be dissolved in aqueous or organic (especially DMSO-based) solutions are used. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

In one preferred embodiment, high throughput screening methods involve providing a combinatorial small organic molecule or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

C. Solid State and Soluble High Throughput Assays

In one embodiment the invention provides soluble assays using a TRIM62 protein disclosed herein, or a cell or tissue expressing such a protein, either naturally occurring or recombinant. In another embodiment, the invention provides solid phase based in vitro assays in a high throughput format, where the TRIM62 protein is attached to a solid phase substrate. Any one of the assays described herein can be adapted for high throughput screening.

In the high throughput assays of the invention, either soluble or solid state, it is possible to screen up to several thousand different modulators or ligands in a single day. This methodology can be used for proteins in vitro, or for cell-based or membrane-based assays comprising a protein of interest. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100-about 1500 different compounds. It is possible to assay many plates per day; assay screens for up to about 6,000, 20,000, 50,000, or more than 100,000 different compounds are possible using the integrated systems of the invention.

For a solid state reaction, the protein of interest or a fragment thereof, e.g., an extracellular domain, or a cell or membrane comprising the protein of interest or a fragment thereof as part of a fusion protein can be bound to the solid state component, directly or indirectly, via covalent or non covalent linkage e.g., via a tag. The tag can be any of a variety of components. In general, a molecule which binds the tag (a tag binder) is fixed to a solid support, and the tagged molecule of interest is attached to the solid support by interaction of the tag and the tag binder.

A number of tags and tag binders can be used, based upon known molecular interactions well described in the literature. For example, where a tag has a natural binder, for example, biotin, protein A, or protein G, it can be used in conjunction with appropriate tag binders (avidin, streptavidin, neutravidin, the Fc region of an immunoglobulin, etc.) Antibodies to molecules with natural binders such as biotin are also widely available and appropriate tag binders; see, SIGMA Immunochemicals 1998 catalogue SIGMA, St. Louis Mo.).

Similarly, any haptenic or antigenic compound can be used in combination with an appropriate antibody to form a tag/tag binder pair. Thousands of specific antibodies are commercially available and many additional antibodies are described in the literature. For example, in one common configuration, the tag is a first antibody and the tag binder is a second antibody which recognizes the first antibody. In addition to antibody-antigen interactions, receptor-ligand interactions are also appropriate as tag and tag-binder pairs. For example, agonists and antagonists of cell membrane receptors (e.g., cell receptor-ligand interactions such as transferrin, c-kit, viral receptor ligands, cytokine receptors, chemokine receptors, interleukin receptors, immunoglobulin receptors and antibodies, the cadherein family, the integrin family, the selectin family, and the like; see, e.g., Pigott & Power, The Adhesion Molecule Facts Book I (1993)). Similarly, toxins and venoms, viral epitopes, hormones (e.g., opiates, steroids, etc.), intracellular receptors (e.g., which mediate the effects of various small ligands, including steroids, thyroid hormone, retinoids and vitamin D; peptides), drugs, lectins, sugars, nucleic acids (both linear and cyclic polymer configurations), oligosaccharides, proteins, phospholipids and antibodies can all interact with various cell receptors.

Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill upon review of this disclosure.

Common linkers such as peptides, polyethers, and the like can also serve as tags, and include polypeptide sequences, such as poly gly sequences of between about 5 and 200 amino acids. Such flexible linkers are known to persons of skill in the art. For example, poly(ethelyne glycol) linkers are available from Shearwater Polymers, Inc. Huntsville, Ala. These linkers optionally have amide linkages, sulfhydryl linkages, or hetero functional linkages.

Tag binders are fixed to solid substrates using any of a variety of methods currently available. Solid substrates are commonly derivatized or functionalized by exposing all or a portion of the substrate to a chemical reagent which fixes a chemical group to the surface which is reactive with a portion of the tag binder. For example, groups which are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The construction of such solid phase biopolymer arrays is well described in the literature. See, e.g., Merrifield, J. Am. Chem. Soc. 85:2149-2154 (1963) (describing solid phase synthesis of, e.g., peptides); Geysen et al., J. Immun. Meth. 102:259-274 (1987) (describing synthesis of solid phase components on pins); Frank & Doring, Tetrahedron 44:60316040 (1988) (describing synthesis of various peptide sequences on cellulose disks); Fodor et al., Science, 251:767-777 (1991); Sheldon et al., Clinical Chemistry 39(4):718-719 (1993); and Kozal et al., Nature Medicine 2(7):753759 (1996) (all describing arrays of biopolymers fixed to solid substrates). Non-chemical approaches for fixing tag binders to substrates include other common methods, such as heat, cross-linking by UV radiation, and the like.

### VII. Diagnostic Assays and Kits for Detection of Cancers Resistant to Anti-ErbB2 Antibody Therapy

The present invention provides diagnostic assays to identify cancers that have increased expression of a member of the epidermal growth factor family, but are resistant to treatment using therapies hat specifically target the epidermal growth factor family member, e.g., a therapeutic antibody or small molecule. The resistant cancers have increased expression of the TRIM62 protein. The increased expression of the TRIM62 protein results in decreased expression of the p27^{Kip1} protein or mislocalization of the p27^{Kip1} to, e.g., the cell cytoplasm. In a preferred embodiment, the resistant cancer overexpresses the ErbB2 protein, e.g., has an amplification of the ErbB2 gene, and also overexpresses the TRIM 62 protein.

A. Assays for TRIM62 Proteins

In one embodiment of the present invention, the presence of TRIM62 protein or nucleic acid is determined by an immunoassay. Enzyme mediated immunoassays such as immunofluorescence assays (IFA), enzyme linked immunosorbent assays (ELISA) and immunoblotting (Western blotting) assays can be readily adapted to accomplish the detection of the TRIM62 proteins. An ELISA method effective for the detection of the TRIM62 protein can, for example, be as follows: (1) bind an anti-TRIM62 antibody or antigen to a substrate; (2) contact the bound antibody with a fluid or tissue sample containing the TRIM62 protein; (3) contact the above with an antibody bound to a detectable moiety (e.g., horseradish peroxidase enzyme or alkaline phosphatase enzyme); (4) contact the above with the substrate for the enzyme; (5) contact the above with a color reagent; (6) observe color change. Those of skill will recognize that the level of TRIM62 protein will be compared to a control sample, e.g., from an unaffected tissue of the test subject or from the same tissue of an unrelated individual.

Another immunologic technique that can be useful in the detection of TRIM62 proteins is the competitive inhibition assay, utilizing monoclonal antibodies (mAbs) specifically reactive with the virus. Briefly, a sample from the subject is reacted with an antibody bound to a substrate (e.g., an ELISA 96-well plate). Excess is thoroughly washed away. A labeled (enzyme-linked, fluorescent, radioactive, etc.) monoclonal antibody is then reacted with the previously reacted parvovirus virus-antibody complex. The amount of inhibition of monoclonal antibody binding is measured relative to a control. mAbs can also be used for detection directly in samples by IFA for mAbs specifically reactive for the antibody-virus complex.

In the diagnostic methods described above, the sample can be taken directly from the subject or in a partially purified form. The antibody specific for the TRIM62 protein (the primary reaction) reacts by binding to the virus. Thereafter, a secondary reaction with an antibody bound to, or labeled with, a detectable moiety can be added to enhance the detection of the primary reaction. Generally, in the secondary reaction, an antibody or other ligand which is reactive, either specifically or nonspecifically with a different binding site (epitope) of the virus will be selected for its ability to react with multiple sites on the complex of antibody and virus. Thus, for example, several molecules of the antibody in the secondary reaction can react with each complex formed by the primary reaction, making the primary reaction more detectable.

The detectable moiety can allow visual detection of a precipitate or a color change, visual detection by microscopy, or automated detection by spectrometry, radiometric measurement or the like. Examples of detectable moieties include fluorescein and rhodamine (for fluorescence microscopy), horseradish peroxidase (for either light or electron microscopy and biochemical detection), biotin-streptavidin (for light or electron microscopy) and alkaline phosphatase (for biochemical detection by color change). The detection methods and moieties used can be selected, for example, from the list above or other suitable examples by the standard criteria applied to such selections (Harlow and Lane, supra).

In solid tumors, immunohistochemical techniques as described above, can be used to assess levels of, e.g., an EGFR family member or a TRIM62 protein.

B. Assays for TRIM62 Nucleic Acids

As described herein, a TRIM62 overexpression may also, or alternatively, be detected based on the level of a TRIM62 RNA or DNA in a biological sample. Primers from TRIM62 can be used for detection of TIRM62 nucleic acids. Any suitable primer can be used to detect the full length TRIM62 nucleic acid or a nucleic acid sub sequence using, e.g., methods described in US 20030104009. For example, the subject nucleic acid compositions can be used as single- or double-stranded probes or primers for the detection of TRIM62 mRNA or cDNA generated from such mRNA, as obtained may be present in a biological sample (e.g., extracts of human cells). The TRIM62 polynucleotides of the invention can also be used to generate additional copies of the polynucleotides, to generate antisense oligonucleotides, and as triple-strand forming oligonucleotides. For example, two oligonucleotide primers can be employed in a polymerase chain reaction (PCR) based assay to amplify a portion of TRIM62 cDNA derived from a biological sample, wherein at least one of the oligonucleotide primers is specific for (i.e., hybridizes to) the HP-4 polynucleotide. The amplified cDNA is then separated and detected using techniques well known in the art, such as gel electrophoresis. Similarly, oligonucleotide probes that specifically hybridize to a TRIM62 polynucleotide can be used in a hybridization assay to detect the presence of the TRIM62 polynucleotide in a biological sample. These and other uses are described in more detail below.

Nucleic acid probes specific to TIRM62 can be generated using the polynucleotide sequences disclosed herein. The probes are preferably at least about 12, 15, 16, 18, 20, 22, 24, or 25 nt fragments of a contiguous sequence of SEQ ID NO: 1 or other polynucleotide sequence encoding a TRIM62 nucleic acid or polypeptide. Nucleic acid probes can be less than about 200 bp, 150 bp, 100 bp, 75 bp, 50 bp, 60 bp, 40 bp, 30 bp, 25 bp 2 kb, 1.5 kb, 1 kb, 0.5 kb, 0.25 kb, 0.1 kb, or 0.05 kb in length. The probes can be produced by, for example, chemical synthesis, PCR amplification, generation from longer polynucleotides using restriction enzymes, or other methods well known in the art.

The polynucleotides of the invention, particularly where used as a probe in a diagnostic assay, can be detectably labeled. Exemplary detectable labels include, but are not limited to, radiolabels, fluorochromes, (e.g., fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein, 6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6-carboxyrho-damine (TAMRA)), radioactive labels, (e.g., ³²P, ³⁵S, and ³H), and the like. The detectable label can involve two stage systems (e.g., biotin-avidin, hapten-anti-hapten antibody, and the like).

The invention also includes solid substrates, such as arrays, comprising any of the polynucleotides described herein. The polynucleotides are immobilized on the arrays using methods known in the art. An array may have one or more different polynucleotides.

Any suitable qualitative or quantitative methods known in the art for detecting specific TRIM62 nucleic acid (e.g., RNA or DNA) can be used. TRIM62 nucleic acid can be detected by, for example, in situ hybridization in tissue sections, using methods that detect single base pair differences between hybridizing nucleic acid (e.g., using the Invader™ technology described in, for example, U.S. Pat. No. 5,846,717), by reverse transcriptase-PCR, or in Northern blots containing poly A⁺ mRNA, and other methods well known in the art.

Using the TRIM62 nucleic acid as a basis, nucleic acid probes (e.g., including oligomers of at least about 8 nucleotides or more) can be prepared, either by excision from recombinant polynucleotides or synthetically, which probes hybridize with the TRIM62 nucleic acid, and thus are useful in detection of TRIM62 in a sample, and identification of resistant cancers. The probes for TRIM62 polynucleotides (natural or derived) are of a length or have a sequence which allows the detection of unique viral sequences by hybridization. While about 6-8 nucleotides may be useful, longer sequences may be preferred, e.g., sequences of about 10-12 nucleotides, or about 20 nucleotides or more.

Nucleic acid probes can be prepared using routine methods, including automated oligonucleotide synthetic methods. A complement to any unique portion of the TRIM62 nucleic acid will be satisfactory. For use as probes, complete complementarity is desirable, though it may be unnecessary as the length of the fragment is increased.

For use of such probes as diagnostics, the biological sample to be analyzed, such as a tumor sample, blood or serum, may be treated, if desired, to extract the nucleic acids contained therein. The resulting nucleic acid from the sample can be subjected to gel electrophoresis or other size separation techniques; alternatively, the nucleic acid sample can be dot blotted without size separation. The probes are usually labeled with a detectable label. Suitable labels, and methods for labeling probes are known in the art, and include, for example, radioactive labels incorporated by nick translation or kinasing, biotin, fluorescent probes, and chemiluminescent probes. The nucleic acids extracted from the sample are then treated with the labeled probe under hybridization conditions of suitable stringencies.

The probes can be made completely complementary to the full length TRIM62 nucleic acid or portion thereof. Therefore, usually high stringency conditions are desirable in order to prevent or at least minimize false positives. The stringency of hybridization is determined by a number of factors during hybridization and during the washing procedure, including temperature, ionic strength, length of time, and concentration of formamide. These factors are outlined in, for example, Sambrook et al., supra).

Non-PCR-based, sequence specific DNA amplification techniques can also be used in the invention to detect TIRM62 sequences. An example of such techniques include, but are not necessarily limited to the Invader assay, see, e.g., Kwiatkowski et al., Mol. Diagn. (1999) 4:353-364. See also U.S. Pat. No. 5,846,717.

A particularly desirable technique may first involve amplification of the target TRIM62 sequences in sera approximately 10,000 fold, e.g., to approximately 10 sequences/mL. This may be accomplished, for example, by the polymerase chain reactions (PCR) technique described which is by Saiki et al., (1986) Nature 324:163-166, by Mullis, U.S. Pat. No. 4,683,195, and by Mullis et al. U.S. Pat. No. 4,683,202. Other amplification methods are well known in the art.

The probes, or alternatively nucleic acid from the samples, can be provided in solution for such assays, or can be affixed to a support (e.g., solid or semi-solid support). Examples of supports that can be used are nitrocellulose (e.g., in membrane or microtiter well form), polyvinyl chloride (e.g., in sheets or microtiter wells), polystyrene latex (e.g., in beads or microtiter plates, polyvinylidine fluoride, diazotized paper, nylon membranes, activated beads, and Protein A beads.

In one embodiment, the probe (or sample nucleic acid) is provided on an array for detection. Arrays can be created by, for example, spotting polynucleotide probes onto a substrate (e.g., glass, nitrocellulose, and the like) in a two-dimensional matrix or array. The probes can be bound to the substrate by either covalent bonds or by non-specific interactions, such as hydrophobic interactions. Samples of polynucleotides can be detectably labeled (e.g., using radioactive or fluorescent labels) and then hybridized to the probes. Double stranded polynucleotides, comprising the labeled sample polynucleotides bound to probe polynucleotides, can be detected once the unbound portion of the sample is washed away. Techniques for constructing arrays and methods of using these arrays are described in EP 799 897; WO 97/29212; WO 97/27317; EP 785 280; WO 97/02357; U.S. Pat. No. 5,593,839; U.S. Pat. No. 5,578,832; EP 728 520; U.S. Pat. No. 5,599,695; EP 721 016; U.S. Pat. No. 5,556,752; WO 95/22058; and U.S. Pat. No. 5,631,734. Arrays are particularly useful where, for example, a single sample is to be analyzed for the presence of two or more nucleic acid target regions, as the probes for each of the target regions, as well as controls (both positive and negative) can be provided on a single array. Arrays thus facilitate rapid and convenience analysis.

C. Kits

The invention further provides diagnostic reagents and kits comprising one or more such reagents for use in a variety of diagnostic assays, including for example, immunoassays such as ELISA and “sandwich”-type immunoassays, as well as nucleic acid assay, e.g., PCR assays. In a related embodiment, the assay is performed in a flow-through or strip test format, wherein the binding agent is immobilized on a membrane, such as nitrocellulose. Such kits can include at least a first peptide, or a first antibody or antigen binding fragment of the invention, a functional fragment thereof, or a cocktail thereof, or a first oligo pair, and means for signal generation. The kit's components can be pre-attached to a solid support, or can be applied to the surface of a solid support when the kit is used. The signal generating means can come pre-associated with an antibody or nucleic acid of the invention or may require combination with one or more components, e.g., buffers, nucleic acids, antibody-enzyme conjugates, enzyme substrates, or the like, prior to use.

Kits can also include additional reagents, e.g., blocking reagents for reducing nonspecific binding to the solid phase surface, washing reagents, enzyme substrates, enzymes, and the like. The solid phase surface may be in the form of microtiter plates, microspheres, or other materials suitable for immobilizing nucleic acids, proteins, peptides, or polypeptides. An enzyme that catalyzes the formation of a chemiluminescent or chromogenic product or the reduction of a chemiluminescent or chromogenic substrate is one such component of the signal generating means. Such enzymes are well known in the art. Where a radiolabel, chromogenic, fluorigenic, or other type of detectable label or detecting means is included within the kit, the labeling agent can be provided either in the same container as the diagnostic or therapeutic composition itself, or may alternatively be placed in a second distinct container means into which this second composition may be placed and suitably aliquoted. Alternatively, the detection reagent and the label can be prepared in a single container means, and in most cases, the kit will also typically include a means for containing the vial(s) in close confinement for commercial sale and/or convenient packaging and delivery.

### VIII. Pharmaceutical Compositions and Administration

Pharmaceutically acceptable carriers are determined in part by the particular composition being administered (e.g., nucleic acid, protein, modulatory compounds or transduced cell), as well as by the particular method used to administer the composition. Accordingly, there are a wide variety of suitable formulations of pharmaceutical compositions of the present invention (see, e.g., Remington's Pharmaceutical Sciences, 17^{th} ed., 1989). Administration can be in any convenient manner, e.g., by injection, oral administration, inhalation, transdermal application, or rectal administration.

Formulations suitable for oral administration can consist of (a) liquid solutions, such as an effective amount of the packaged nucleic acid suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

The compound of choice, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration and intravenous administration are the preferred methods of administration. The formulations of commends can be presented in unit-dose or multi-dose sealed containers, such as ampules and vials.

Injection solutions and suspensions can be prepared from sterile powders, granules, and tablets of the kind previously described. Cells transduced by nucleic acids for ex vivo therapy can also be administered intravenously or parenterally as described above.

The dose administered to a patient, in the context of the present invention should be sufficient to effect a beneficial therapeutic response in the patient over time. The dose will be determined by the efficacy of the particular vector employed and the condition of the patient, as well as the body weight or surface area of the patient to be treated. The size of the dose also will be determined by the existence, nature, and extent of any adverse side-effects that accompany the administration of a particular vector, or transduced cell type in a particular patient.

In determining the effective amount of the vector to be administered in the treatment or prophylaxis of conditions owing to diminished or aberrant expression of the protein, the physician evaluates circulating plasma levels of the vector, vector toxicities, progression of the disease, and the production of anti-vector antibodies. In general, the dose equivalent of a naked nucleic acid from a vector is from about 1 μg to 100 μg for a typical 70 kilogram patient, and doses of vectors which include a retroviral particle are calculated to yield an equivalent amount of therapeutic nucleic acid.

For administration, compounds and transduced cells of the present invention can be administered at a rate determined by the LD-50 of the inhibitor, vector, or transduced cell type, and the side-effects of the inhibitor, vector or cell type at various concentrations, as applied to the mass and overall health of the patient. Administration can be accomplished via single or divided doses.

### IX. Molecular Biology and Biochemical Techniques

A. Isolation of Nucleic Acids Encoding TRIM62 Polypeptides

Nucleic acids that encode TRIM62 polypeptides include nucleic acids that encode the full-length, naturally occurring TRIM62 polypeptides described above and enzymatically active truncations of those sequences. The TRIM62 polypeptides of the invention catalyze the transfer of a sialic acid moiety from a donor substrate to an acceptor substrate and assays to measure that activity are disclosed herein.

Nucleic acids that encode additional TRIM62 polypeptides based on the information disclosed herein, and methods of obtaining such nucleic acids, are known to those of skill in the art. Suitable nucleic acids (e.g., cDNA, genomic, or subsequences (probes)) can be cloned, or amplified by in vitro methods such as the polymerase chain reaction (PCR), the ligase chain reaction (LCR), the transcription-based amplification system (TAS), or the self-sustained sequence replication system (SSR). A wide variety of cloning and in vitro amplification methodologies are well-known to persons of skill. Examples of these techniques and instructions sufficient to direct persons of skill through many cloning exercises are found in Berger and Kimmel, Guide to Molecular Cloning Techniques, Methods in Enzymology 152 Academic Press, Inc., San Diego, Calif. (Berger); Sambrook et al. (1989) Molecular Cloning—A Laboratory Manual (2nd ed.) Vol. 1-3, Cold Spring Harbor Laboratory, Cold Spring Harbor Press, NY, (Sambrook et al.); Current Protocols in Molecular Biology, F. M. Ausubel et al., eds., Current Protocols, a joint venture between Greene Publishing Associates, Inc. and John Wiley & Sons, Inc., (1994 Supplement) (Ausubel); Cashion et al., U.S. Pat. No. 5,017,478; and Can, European Patent No. 0 246 864.

Standard molecular biology methods, e.g., PCR, can be used to generate truncations of any known TRIM62 sequence.

A DNA that encodes a TRIM62 polypeptide, or a subsequence or truncation thereof, can be prepared by any suitable method described above, including, for example, cloning and restriction of appropriate sequences with restriction enzymes. In one embodiment, nucleic acids encoding TRIM62 polypeptides are isolated by routine cloning methods. A nucleotide sequence encoding a TRIM62 polypeptide is, for example, a nucleic acid sequence coding for the TRIM62 of SEQ ID NO:1. Nucleic acid sequences can be used to provide probes that specifically hybridize to a gene encoding a TRIM62 polypeptide in a genomic DNA sample; or to an mRNA, encoding a TRIM62 polypeptide in a total RNA sample (e.g., in a Southern or Northern blot). Once the target nucleic acid encoding a TRIM62 polypeptide is identified, it can be isolated according to standard methods known to those of skill in the art (see, e.g., Sambrook et al. (1989) Molecular Cloning: A Laboratory Manual, 2nd Ed., Vols. 1-3, Cold Spring Harbor Laboratory; Berger and Kimmel (1987) Methods in Enzymology, Vol. 152: Guide to Molecular Cloning Techniques, San Diego: Academic Press, Inc.; or Ausubel et al. (1987) Current Protocols in Molecular Biology, Greene Publishing and Wiley-Interscience, New York). Further, the isolated nucleic acids can be cleaved with restriction enzymes to create nucleic acids encoding the full-length a TRIM62 polypeptide, or subsequences or truncation variants thereof, e.g., truncations containing subsequences encoding at least a subsequence of a catalytic domain of a TRIM62 polypeptide. These restriction enzyme fragments, encoding a TRIM62 polypeptide or subsequences thereof, can then be ligated.

A nucleic acid encoding a TRIM62 polypeptide, or a subsequence thereof, can be characterized by assaying for the expressed product. Assays based on the detection of the physical, chemical, or immunological properties of the expressed protein can be used. For example, one can identify a cloned TRIM62 polypeptide, by the presence of E3 ligase activity.

Also, a nucleic acid encoding a TRIM62 polypeptide, or a subsequence thereof, can be chemically synthesized. Suitable methods include the phosphotriester method of Narang et al. (1979) Meth. Enzymol. 68: 90-99; the phosphodiester method of Brown et al. (1979) Meth. Enzymol. 68: 109-151; the diethylphosphoramidite method of Beaucage et al. (1981) Tetra. Lett., 22: 1859-1862; and the solid support method of U.S. Pat. No. 4,458,066. Chemical synthesis produces a single stranded oligonucleotide. This can be converted into double stranded DNA by hybridization with a complementary sequence, or by polymerization with a DNA polymerase using the single strand as a template. One of skill recognizes that while chemical synthesis of DNA is often limited to sequences of about 100 bases, longer sequences may be obtained by the ligation of shorter sequences.

Nucleic acids encoding TRIM62 polypeptides, or subsequences thereof, can be cloned using DNA amplification methods such as polymerase chain reaction (PCR). Thus, for example, the nucleic acid sequence or subsequence is PCR amplified, using a sense primer containing one restriction enzyme site (e.g., NdeI) and an antisense primer containing another restriction enzyme site (e.g., HindIII). This will produce a nucleic acid encoding the desired TRIM62 polypeptide or a subsequence and having terminal restriction enzyme sites. This nucleic acid can then be easily ligated into an expression vector having the appropriate corresponding restriction enzyme sites. Suitable PCR primers can be determined by one of skill in the art using the sequence information provided in GenBank® or other sources. Appropriate restriction enzyme sites can also be added to the nucleic acid encoding the TRIM62 polypeptide or a protein subsequence thereof by site-directed mutagenesis. The plasmid containing the TRIM62 polypeptide-encoding nucleotide sequence or subsequence is cleaved with the appropriate restriction endonuclease and then ligated into an appropriate vector for amplification and/or expression according to standard methods. Examples of techniques sufficient to direct persons of skill through in vitro amplification methods are found in Berger, Sambrook, and Ausubel, as well as Mullis et al., (1987) U.S. Pat. No. 4,683,202; PCR Protocols A Guide to Methods and Applications (Innis et al., eds) Academic Press Inc. San Diego, Calif. (1990) (Innis); Amheim & Levinson (Oct. 1, 1990) C&EN 36-47; The Journal Of NIH Research (1991) 3: 81-94; (Kwoh et al. (1989) Proc. Natl. Acad. Sci. USA 86: 1173; Guatelli et al. (1990) Proc. Natl. Acad. Sci. USA 87, 1874; Lomell et al. (1989) J. Clin. Chem., 35: 1826; Landegren et al., (1988) Science 241: 1077-1080; Van Brunt (1990) Biotechnology 8: 291-294; Wu and Wallace (1989) Gene 4: 560; and Barringer et al. (1990) Gene 89: 117.

Other physical properties of a recombinant TRIM62 polypeptide expressed from a particular nucleic acid, can be compared to properties of known TRIM62 polypeptides to provide another method of identifying suitable sequences or domains of the TRIM62 polypeptide that are determinants of acceptor substrate specificity and/or catalytic activity. Alternatively, a putative TRIM62 polypeptide can be mutated, and its role as a TRIM62, or the role of particular sequences or domains established by detecting a variation in the structure of a carbohydrate normally produced by the unmutated, naturally-occurring, or control TRIM62 polypeptide. Those of skill will recognize that mutation or modification of TRIM62 polypeptide of the invention can be facilitated by molecular biology techniques to manipulate the nucleic acids encoding the TRIM62 polypeptide, e.g., PCR.

B. Expressing TRIM62 Polypeptides in Host Cells

TRIM62 proteins of the invention can be expressed in a variety of host cells, including E. coli, other bacterial hosts, and yeast. The host cells are preferably microorganisms, such as, for example, yeast cells, bacterial cells, or filamentous fungal cells. Examples of suitable host cells include, for example, Azotobacter sp. (e.g., A. vinelandii), Pseudomonas sp., Rhizobium sp., Erwinia sp., Escherichia sp. (e.g., E. coli), Bacillus, Pseudomonas, Proteus, Salmonella, Serratia, Shigella, Rhizobia, Vitreoscilla, Paracoccus and Klebsiella sp., among many others. The cells can be of any of several genera, including Saccharomyces (e.g., S. cerevisiae), Candida (e.g., C. utilis, C. parapsilosis, C. krusei, C. versatilis, C. lipolytica, C. zeylanoides, C. guilliermondii, C. albicans, and C. humicola), Pichia (e.g., P. farinosa and P. ohmeri), Torulopsis (e.g., T. candida, T. sphaerica, T. xylinus, T. famata, and T. versatilis), Debaryomyces (e.g., D. subglobosus, D. cantarellii, D. globosus, D. hansenii, and D. japonicus), Zygosaccharomyces (e.g., Z. rouxii and Z. bailiff), Kluyveromyces (e.g., K. marxianus), Hansenula (e.g., H. anomala and H. jadinii), and Brettanomyces (e.g., B. lambicus and B. anomalus). Examples of useful bacteria include, but are not limited to, Escherichia, Enterobacter, Azotobacter, Erwinia, Klebsielia, Bacillus, Pseudomonas, Proteus, and Salmonella.

Typically, the polynucleotide that encodes the TRIM62 polypeptides is placed under the control of a promoter that is functional in the desired host cell. An extremely wide variety of promoters are well known, and can be used in the expression vectors of the invention, depending on the particular application. Ordinarily, the promoter selected depends upon the cell in which the promoter is to be active. Other expression control sequences such as ribosome binding sites, transcription termination sites and the like are also optionally included. Constructs that include one or more of these control sequences are termed “expression cassettes.” Accordingly, the invention provides expression cassettes into which the nucleic acids that encode fusion proteins are incorporated for high level expression in a desired host cell.

Expression control sequences that are suitable for use in a particular host cell are often obtained by cloning a gene that is expressed in that cell. Commonly used prokaryotic control sequences, which are defined herein to include promoters for transcription initiation, optionally with an operator, along with ribosome binding site sequences, include such commonly used promoters as the beta-lactamase (penicillinase) and lactose (lac) promoter systems (Change et al., Nature (1977) 198:1056), the tryptophan (trp) promoter system (Goeddel et al., Nucleic Acids Res. (1980) 8:4057), the tac promoter (DeBoer, et al., Proc. Natl. Acad. Sci. U.S.A. (1983) 80:21-25); and the lambda-derived P_{L} promoter and N-gene ribosome binding site (Shimatake et al., Nature (1981) 292:128). The particular promoter system is not critical to the invention, any available promoter that functions in prokaryotes can be used.

For expression of TRIM62 proteins in prokaryotic cells other than E. coli, a promoter that functions in the particular prokaryotic species is required. Such promoters can be obtained from genes that have been cloned from the species, or heterologous promoters can be used. For example, the hybrid trp-lac promoter functions in Bacillus in addition to E. coli.

A ribosome binding site (RBS) is conveniently included in the expression cassettes of the invention. An RBS in E. coli, for example, consists of a nucleotide sequence 3-9 nucleotides in length located 3-11 nucleotides upstream of the initiation codon (Shine and Dalgarno, Nature (1975) 254:34; Steitz, In Biological regulation and development: Gene expression (ed. R. F. Goldberger), vol. 1, p. 349, 1979, Plenum Publishing, NY).

For expression of the TRIM62 proteins in yeast, convenient promoters include GAL1-10 (Johnson and Davies (1984) Mol. Cell. Biol. 4:1440-1448) ADH2 (Russell et al. (1983) J. Biol. Chem. 258:2674-2682), PHO5 (EMBO J. (1982) 6:675-680), and MFα (Herskowitz and Oshima (1982) in The Molecular Biology of the Yeast Saccharomyces (eds. Strathern, Jones, and Broach) Cold Spring Harbor Lab., Cold Spring Harbor, N.Y., pp. 181-209). Another suitable promoter for use in yeast is the ADH2/GAPDH hybrid promoter as described in Cousens et al., Gene 61:265-275 (1987). For filamentous fungi such as, for example, strains of the fungi Aspergillus (McKnight et al., U.S. Pat. No. 4,935,349), examples of useful promoters include those derived from Aspergillus nidulans glycolytic genes, such as the ADH3 promoter (McKnight et al., EMBO J. 4:2093 2099 (1985)) and the tpiA promoter. An example of a suitable terminator is the ADH3 terminator (McKnight et al., supra).

Either constitutive or regulated promoters can be used in the present invention. Regulated promoters can be advantageous because the host cells can be grown to high densities before expression of the fusion proteins is induced. High level expression of heterologous proteins slows cell growth in some situations. An inducible promoter is a promoter that directs expression of a gene where the level of expression is alterable by environmental or developmental factors such as, for example, temperature, pH, anaerobic or aerobic conditions, light, transcription factors and chemicals. Such promoters are referred to herein as “inducible” promoters, which allow one to control the timing of expression of the glycosyltransferase or enzyme involved in nucleotide sugar synthesis. For E. coli and other bacterial host cells, inducible promoters are known to those of skill in the art. These include, for example, the lac promoter, the bacteriophage lambda P_{L} promoter, the hybrid trp-lac promoter (Amann et al. (1983) Gene 25:167; de Boer et al. (1983) Proc. Natl. Acad. Sci. USA 80: 21), and the bacteriophage T7 promoter (Studier et al. (1986) J. Mol. Biol. 189:113-130; Tabor et al. (1985) Proc. Natl. Acad. Sci. USA 82:1074-8). These promoters and their use are discussed in Sambrook et al., supra. A particularly preferred inducible promoter for expression in prokaryotes is a dual promoter that includes a tac promoter component linked to a promoter component obtained from a gene or genes that encode enzymes involved in galactose metabolism (e.g., a promoter from a UDPgalactose 4-epimerase gene (galE)). The dual tac-gal promote is described in WO98/20111.

A construct that includes a polynucleotide of interest operably linked to gene expression control signals that, when placed in an appropriate host cell, drive expression of the polynucleotide is termed an “expression cassette.” Expression cassettes that encode the fusion proteins of the invention are often placed in expression vectors for introduction into the host cell. The vectors typically include, in addition to an expression cassette, a nucleic acid sequence that enables the vector to replicate independently in one or more selected host cells. Generally, this sequence is one that enables the vector to replicate independently of the host chromosomal DNA, and includes origins of replication or autonomously replicating sequences. Such sequences are well known for a variety of bacteria. For instance, the origin of replication from the plasmid pBR322 is suitable for most Gram-negative bacteria. Alternatively, the vector can replicate by becoming integrated into the host cell genomic complement and being replicated as the cell undergoes DNA replication. A preferred expression vector for expression of the enzymes is in bacterial cells is pTGK, which includes a dual tac-gal promoter and is described in WO98/20111.

The construction of polynucleotide constructs generally requires the use of vectors able to replicate in bacteria. A plethora of kits are commercially available for the purification of plasmids from bacteria (see, for example, EasyPrepJ®, FlexiPrepJ®, both from Pharmacia Biotech; StrataCleanJ®, from Stratagene; and, QIAexpress® Expression System, Qiagen). The isolated and purified plasmids can then be further manipulated to produce other plasmids, and used to transfect cells. Cloning in Streptomyces or Bacillus is also possible.

Selectable markers are often incorporated into the expression vectors used to express the polynucleotides of the invention. These genes can encode a gene product, such as a protein, necessary for the survival or growth of transformed host cells grown in a selective culture medium. Host cells not transformed with the vector containing the selection gene will not survive in the culture medium. Typical selection genes encode proteins that confer resistance to antibiotics or other toxins, such as ampicillin, neomycin, kanamycin, chloramphenicol, or tetracycline. Alternatively, selectable markers may encode proteins that complement auxotrophic deficiencies or supply critical nutrients not available from complex media, e.g., the gene encoding D-alanine racemase for Bacilli. Often, the vector will have one selectable marker that is functional in, e.g., E. coli, or other cells in which the vector is replicated prior to being introduced into the host cell. A number of selectable markers are known to those of skill in the art and are described for instance in Sambrook et al., supra.

Construction of suitable vectors containing one or more of the above listed components employs standard ligation techniques as described in the references cited above. Isolated plasmids or DNA fragments are cleaved, tailored, and re-ligated in the form desired to generate the plasmids required. To confirm correct sequences in plasmids constructed, the plasmids can be analyzed by standard techniques such as by restriction endonuclease digestion, and/or sequencing according to known methods. Molecular cloning techniques to achieve these ends are known in the art. A wide variety of cloning and in vitro amplification methods suitable for the construction of recombinant nucleic acids are well-known to persons of skill. Examples of these techniques and instructions sufficient to direct persons of skill through many cloning exercises are found in Berger and Kimmel, Guide to Molecular Cloning Techniques, Methods in Enzymology, Volume 152, Academic Press, Inc., San Diego, Calif. (Berger); and Current Protocols in Molecular Biology, F. M. Ausubel et al., eds., Current Protocols, a joint venture between Greene Publishing Associates, Inc. and John Wiley & Sons, Inc., (1998 Supplement) (Ausubel).

A variety of common vectors suitable for use as starting materials for constructing the expression vectors of the invention are well known in the art. For cloning in bacteria, common vectors include pBR322 derived vectors such as pBLUESCRIPT™, and λ-phage derived vectors. In yeast, vectors include Yeast Integrating plasmids (e.g., YIp5) and Yeast Replicating plasmids (the YRp series plasmids) and pGPD-2. Expression in mammalian cells can be achieved using a variety of commonly available plasmids, including pSV2, pBC12BI, and p91023, as well as lytic virus vectors (e.g., vaccinia virus, adenovirus, and baculovirus), episomal virus vectors (e.g., bovine papillomavirus), and retroviral vectors (e.g., murine retroviruses).

The methods for introducing the expression vectors into a chosen host cell are not particularly critical, and such methods are known to those of skill in the art. For example, the expression vectors can be introduced into prokaryotic cells, including E. coli, by calcium chloride transformation, and into eukaryotic cells by calcium phosphate treatment or electroporation. Other transformation methods are also suitable.

Translational coupling may be used to enhance expression. The strategy uses a short upstream open reading frame derived from a highly expressed gene native to the translational system, which is placed downstream of the promoter, and a ribosome binding site followed after a few amino acid codons by a termination codon. Just prior to the termination codon is a second ribosome binding site, and following the termination codon is a start codon for the initiation of translation. The system dissolves secondary structure in the RNA, allowing for the efficient initiation of translation. See Squires, et. al. (1988), J. Biol. Chem. 263: 16297-16302.

The TRIM62 polypeptides can be expressed intracellularly, or can be secreted from the cell. Intracellular expression often results in high yields. If necessary, the amount of soluble, active fusion protein may be increased by performing refolding procedures (see, e.g., Sambrook et al., supra.; Marston et al., Bio/Technology (1984) 2:800; Schoner et al., Bio/Technology (1985) 3:151). In embodiments in which the TRIM62 polypeptides are secreted from the cell, either into the periplasm or into the extracellular medium, the DNA sequence is linked to a cleavable signal peptide sequence. The signal sequence directs translocation of the fusion protein through the cell membrane. An example of a suitable vector for use in E. coli that contains a promoter-signal sequence unit is pTA1529, which has the E. coli phoA promoter and signal sequence (see, e.g., Sambrook et al., supra.; Oka et al., Proc. Natl. Acad. Sci. USA (1985) 82:7212; Talmadge et al., Proc. Natl. Acad. Sci. USA (1980) 77:3988; Takahara et al., J. Biol. Chem. (1985) 260:2670). In another embodiment, the TRIM62 proteins are fused to a subsequence of protein A or bovine serum albumin (BSA), for example, to facilitate purification, secretion, or stability.

The TRIM62 polypeptides of the invention can also be further linked to other bacterial proteins. This approach often results in high yields, because normal prokaryotic control sequences direct transcription and translation. In E. coli, lacZ fusions are often used to express heterologous proteins. Suitable vectors are readily available, such as the pUR, pEX, and pMR100 series (see, e.g., Sambrook et al., supra.). For certain applications, it may be desirable to cleave the non-TRIM62 amino acids from the fusion protein after purification. This can be accomplished by any of several methods known in the art, including cleavage by cyanogen bromide, a protease, or by Factor X, (see, e.g., Sambrook et al., supra.; Itakura et al., Science (1977) 198:1056; Goeddel et al., Proc. Natl. Acad. Sci. USA (1979) 76:106; Nagai et al., Nature (1984) 309:810; Sung et al., Proc. Natl. Acad. Sci. USA (1986) 83:561). Cleavage sites can be engineered into the gene for the fusion protein at the desired point of cleavage.

C. Purification of TRIM62 Polypeptides

The TRIM62 proteins of the present invention can be expressed, e.g., as intracellular proteins or as proteins that are secreted from the cell, and can be used in this form, in the methods of the present invention. For example, a crude cellular extract containing the expressed intracellular or secreted TRIM62 polypeptide can used in the methods of the present invention.

Alternatively, the TRIM62 polypeptide can be purified according to standard procedures of the art, including ammonium sulfate precipitation, affinity columns, column chromatography, gel electrophoresis and the like (see, generally, R. Scopes, Protein Purification, Springer-Verlag, N.Y. (1982), Deutscher, Methods in Enzymology Vol. 182: Guide to Protein Purification., Academic Press, Inc. N.Y. (1990)). Substantially pure compositions of at least about 70, 75, 80, 85, 90% homogeneity are preferred, and 92, 95, 98 to 99% or more homogeneity are most preferred. The purified proteins may also be used, e.g., as immunogens for antibody production.

To facilitate purification of the TRIM62 polypeptides of the invention, the nucleic acids that encode the proteins can also include a coding sequence for an epitope or “tag” for which an affinity binding reagent is available, i.e. a purification tag. Examples of suitable epitopes include the myc and V-5 reporter genes; expression vectors useful for recombinant production of fusion proteins having these epitopes are commercially available (e.g., Invitrogen (Carlsbad Calif.) vectors pcDNA3.1/Myc-His and pcDNA3.1/V5-His are suitable for expression in mammalian cells). Additional expression vectors suitable for attaching a tag to a TRIM62 polypeptide of the invention, and corresponding detection systems are known to those of skill in the art, and several are commercially available (e.g., “FLAG” (Kodak, Rochester N.Y.). Another example of a suitable tag is a polyhistidine sequence, which is capable of binding to metal chelate affinity ligands. Typically, six adjacent histidines are used, although one can use more or less than six. Suitable metal chelate affinity ligands that can serve as the binding moiety for a polyhistidine tag include nitrilo-tri-acetic acid (NTA) (Hochuli, (1990) “Purification of recombinant proteins with metal chelating adsorbents” In Genetic Engineering: Principles and Methods, Setlow, Ed., Plenum Press, NY; commercially available from Qiagen (Santa Clarita, Calif.)). Other purification or epitope tags include, e.g., AU1, AU5, DDDDK (EC5), E tag, E2 tag, Glu-Glu, a 6 residue peptide, EYMPME, derived from the Polyoma middle T protein, HA, HSV, IRS, KT3, S tage, S1 tag, T7 tag, V5 tag, VSV-G, β-galactosidase, Gal4, green fluorescent protein (GFP), luciferase, protein C, protein A, cellulose binding protein, GST (glutathione S-transferase), a step-tag, Nus-S, PPI-ases, Pfg 27, calmodulin binding protein, dsb A and fragments thereof, and granzyme B. Epitope peptides and antibodies that bind specifically to epitope sequences are commercially available from, e.g., Covance Research Products, Inc.; Bethyl Laboratories, Inc.; Abcam Ltd.; and Novus Biologicals, Inc.

Purification tags also include maltose binding domains and starch binding domains. Proteins comprising purification tags can be purified using a binding partner that binds the purification tag, e.g., antibodies to the purification tag, nickel or cobalt ions or resins, and amylose, maltose, or a cyclodextrin. Purification tags also include starch binding domains, E. coli thioredoxin domains (vectors and antibodies commercially available from e.g., Santa Cruz Biotechnology, Inc. and Alpha Diagnostic International, Inc.), and the carboxy-terminal half of the SUMO protein (vectors and antibodies commercially available from e.g., Life Sensors Inc.). Starch binding domains, such as a maltose binding domain from E. coli and SBD (starch binding domain) from an amylase of A. niger, are described in WO 99/15636, herein incorporated by reference. Affinity purification of a fusion protein comprising a starch binding domain using a betacyclodextrin (BCD)-derivatized resin is described in WO 2005/014779, herein incorporated by reference in its entirety. In some embodiments, a TRIM62 polypeptide comprises more than one purification or epitope tag.

Other haptens that are suitable for use as tags are known to those of skill in the art and are described, for example, in the Handbook of Fluorescent Probes and Research Chemicals (6th Ed., Molecular Probes, Inc., Eugene Oreg.). For example, dinitrophenol (DNP), digoxigenin, barbiturates (see, e.g., U.S. Pat. No. 5,414,085), and several types of fluorophores are useful as haptens, as are derivatives of these compounds. Kits are commercially available for linking haptens and other moieties to proteins and other molecules. For example, where the hapten includes a thiol, a heterobifunctional linker such as SMCC can be used to attach the tag to lysine residues present on the capture reagent.

One of skill would recognize that modifications can be made to the catalytic or functional domains of the TRIM62 polypeptide without diminishing their biological activity. Some modifications may be made to facilitate the cloning, expression, or incorporation of the catalytic domain into a fusion protein. Such modifications are well known to those of skill in the art and include, for example, the addition of codons at either terminus of the polynucleotide that encodes the catalytic domain to provide, for example, a methionine added at the amino terminus to provide an initiation site, or additional amino acids (e.g., poly His) placed on either terminus to create conveniently located restriction enzyme sites or termination codons or purification sequences.

It must be noted that as used herein and in the appended claims, the singular forms “a”, “and”, and “the” include plural referents unless the context clearly dictates otherwise. Thus, for example, reference to “a nucleic acid” includes a plurality of such nucleic acids and reference to “the polypeptide” includes reference to one or more polypeptides and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed. All citations are incorporated herein by reference.

### EXAMPLES

### Example 1

### Identification of Regulators of p27

A high throughput siRNA screen was performed to identify regulators of p27^{Kip1}. A p27^{Kip1} reporter construct was stably integrated into the genome of the human glioblastoma cell line U251. The reporter construct included a p27^{Kip1} mRNA fused in frame to a luciferase nucleic acid to create a fusion protein with conveniently measurable activity. The siRNA library targeted most known regulators of ubiquitin-dependent protein degradation. Each target nucleic acid was targeted by a pool of three specific siRNAs.

U251 cells with the p27^{Kip1} reporter construct were seeded into ninety-six well plates. An automated delivery system was used to administer the siRNA library to the cells. Luciferase activity was measured in situ. Cells with high levels of luciferase activity were analyzed further and siRNA targets were identified. One of the siRNA targets was an E3 ubiquitin ligase, TRIM62.

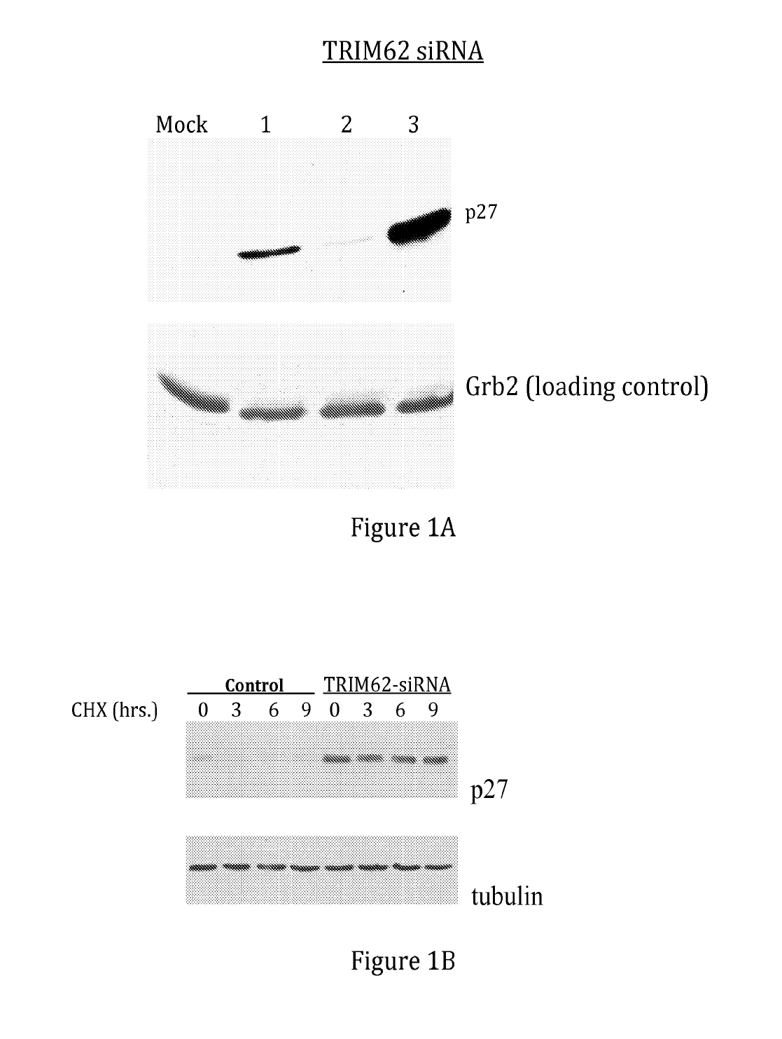

The effect of TRIM62 siRNA treatment on endogenous p27^{Kip1} protein expression was assessed. FIGS. 1A and B demonstrate the effect of transient reduction of TRIM62 expression in HeLa cells, a transformed cell line. TRIM62 siRNA was transfected into HeLa cells and transiently expressed. Cell extracts were prepared and proteins were separated using SDS-PAGE. After transfer of the separated proteins to a nylon filter, p27^{Kip1} levels were assayed by Western blotting using a p27^{Kip1} specific antibody. FIG. 1A shows the results with three different TRIM62 siRNA molecules, in lanes 1, 2, and 3. A control sample, labeled mock, was transfected with an unrelated siRNA. Elimination of TRIM62 expression using TRIM62 siRNA's 1 and 3 greatly increased the levels of p27^{Kip1} protein as measured by Western blotting. As a control, levels of the unrelated Grb2 protein were assessed by reprobing the blot with a Grb2 specific antibody.

In FIG. 1B, HeLa cells were treated with cyclohexamide (CXH) over a period of nine hours. Control samples were transfected with an unrelated siRNA. Experimental samples were transfected with a TRIM62 specific siRNA. As a control, levels of the unrelated tubulin protein were assessed by reprobing the blot with a tubulin specific antibody. In the presence of TRIM62 siRNA, p27^{Kip1} levels were higher than a control at time zero and remained high for nine hours after cycloheximide treatment.

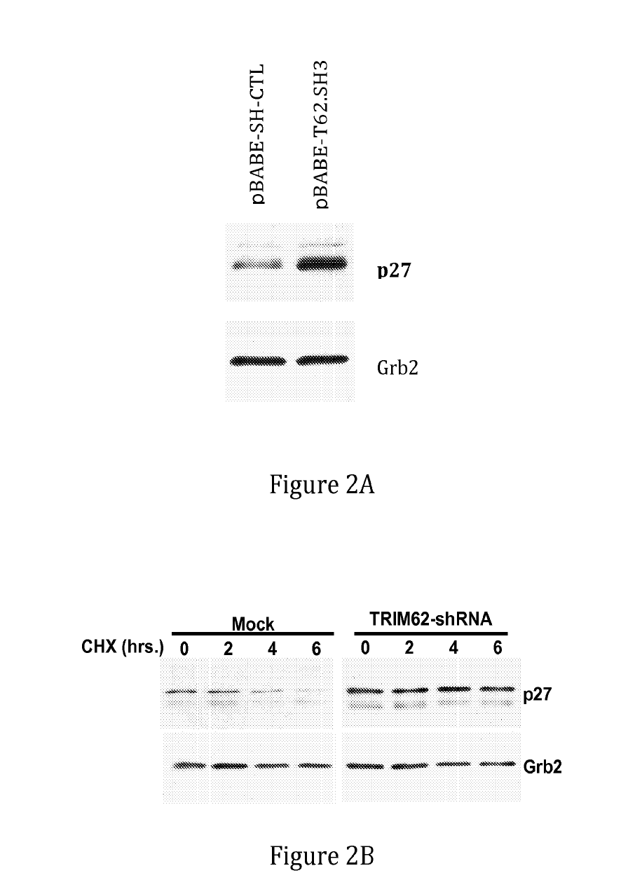

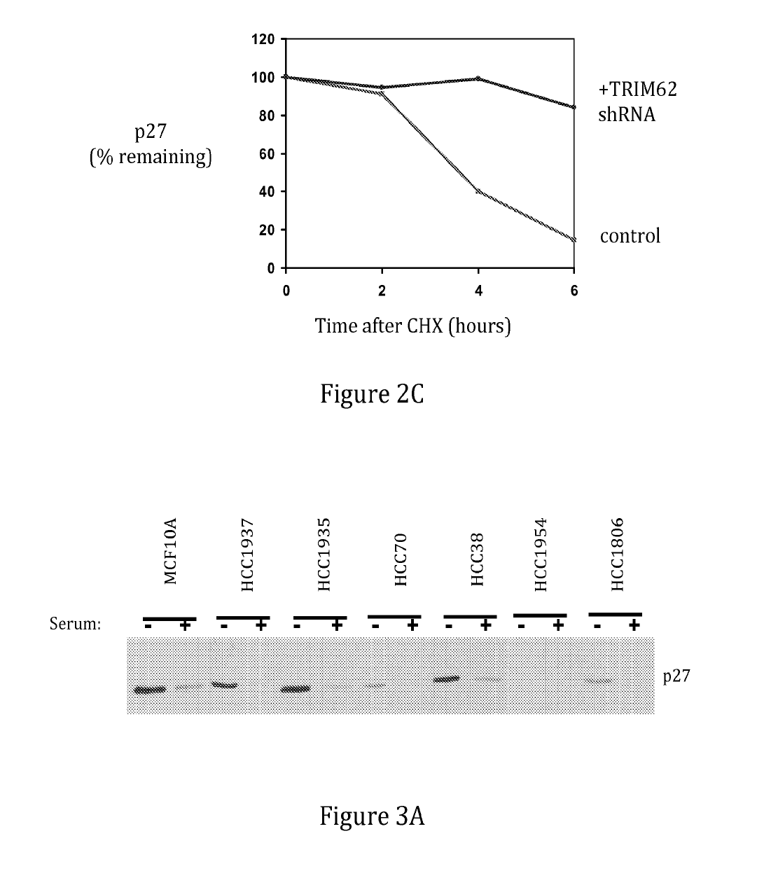

FIGS. 2A-C demonstrate the effect of reducing expression of the TRIM62 nucleic acid and protein in U251 cells, a transformed cell line derived from a glioblastoma. U251 cells were stably transfected to express a TRIM62 siRNA. Cell extracts were prepared and proteins were separated using SDS-PAGE. After transfer of the separated proteins to a nylon filter, p27 levels were assayed by Western blotting using a p27^{Kip1} specific antibody. FIG. 2A shows the results with a TRIM62 siRNA molecule, in lane 2. A control sample, labeled pBABE-SH-CTL, was transfected with an unrelated siRNA. Elimination of TRIM62 expression using TRIM62 siRNA greatly increased the levels of p27^{Kip1} protein as measured by Western blotting. As a control, levels of the unrelated Grb2 protein were assessed by reprobing the blot with a Grb2 specific antibody.

In FIG. 2B, U251 cells were treated with cyclohexamide over a period of six hours. Control samples (mock) were transfected with an unrelated siRNA. Experimental samples were transfected with a TRIM62 specific siRNA. As a control, levels of the unrelated tubulin protein were assessed by reprobing the blot with a tubulin specific antibody. FIG. 2C shows the time course of p27^{Kip1} expression after CHX treatment in the presence and absence of a TRIM62 siRNA. In the presence of TRIM62 siRNA, p27^{Kip1} levels were higher than a control at time zero and remained high for six hours after cycloheximide treatment.

### Example 2

### ErbB2 Status Affects TRIM62 Regulation of p27^{Kip1} Levels in Breast Cancer Cells

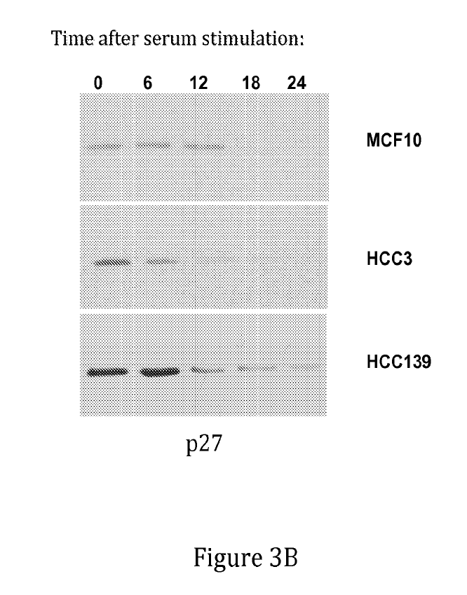

p27^{Kip1} protein expression was assayed in breast cancer cell lines. First, breast cancer cell lines with unmodified ErbB2 activity were assayed. FIGS. 3A and 3B show the effect of serum addition on p27^{Kip1} levels after a G1 cell cycle block in breast cancer cell lines that do not overexpress the ErbB2 protein. p27^{Kip1} levels were assayed by Western blotting using a p27^{Kip1} specific antibody. FIG. 3A shows a single time point and FIG. 3B shows a time course of p27^{Kip1} protein levels during a twenty-four hour period following addition of serum to the serum-starved cells. p27^{Kip1} was expressed in the serum starved cells, but expression levels decreased after addition of serum. In HCC38 cells, p27^{Kip1} levels decreased after serum addition and were almost undetectable between twelve and twenty-four hours later.

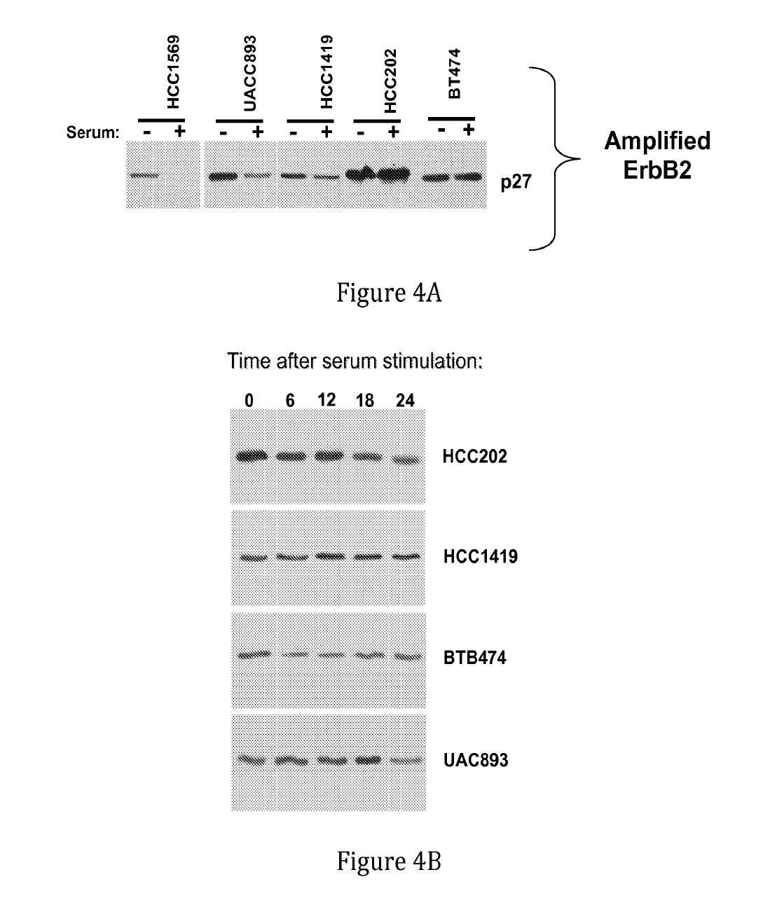

Experiments were repeated in breast cancer cell lines that have an amplification of the ErbB2 gene and increased ErbB2 activity. FIGS. 4A and 4B show the effect of serum addition on p27^{Kip1} levels after a G1 cell cycle block in those cell lines. p27^{Kip1} levels were again assayed by Western blotting using a p27 specific antibody. FIG. 4A shows the effect of serum addition on p27 protein expression after serum starvation. Little difference in p27^{Kip1} levels was detected at this single time point. FIG. 4B shows a time course of p27^{Kip1} protein levels during a twenty-four hour period following addition of serum to serum-starved cells. p27^{Kip1} levels did not decline during the twenty-four hour period. Intact cells were stained with p27^{Kip1} antibody. p27^{Kip1} in the ErbB2 amplified cells was predominantly found in the cytoplasm. Data not shown. This is in contrast with earlier studies, which showed that most p27^{Kip1} protein is localized to the cell nucleus.

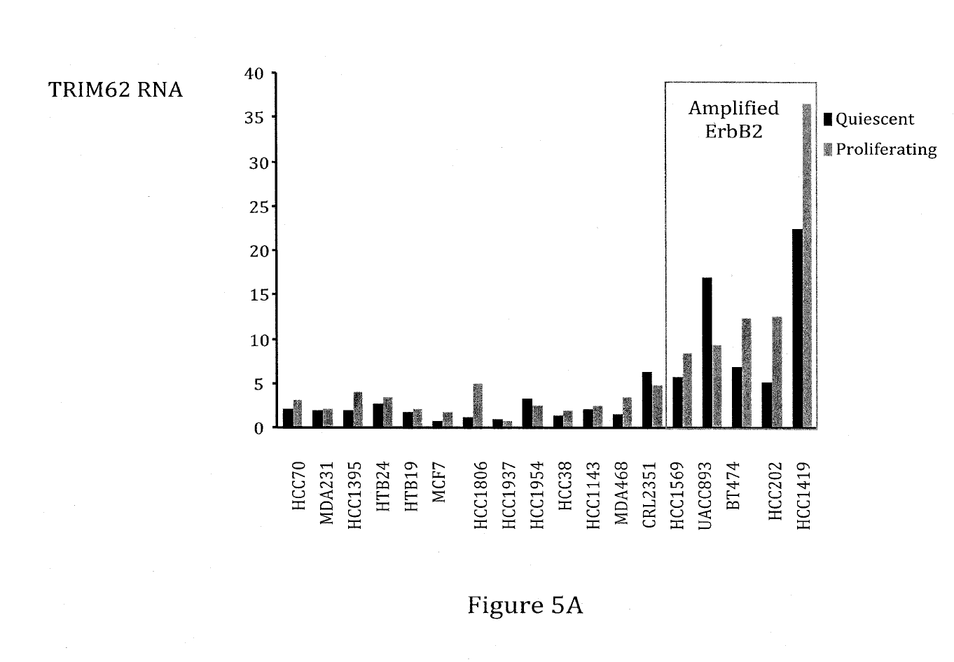

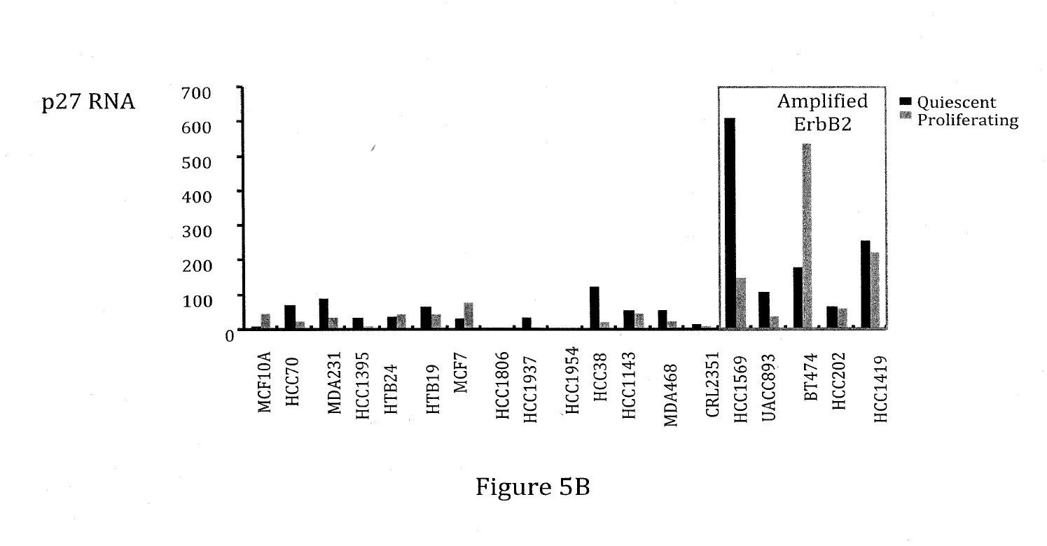

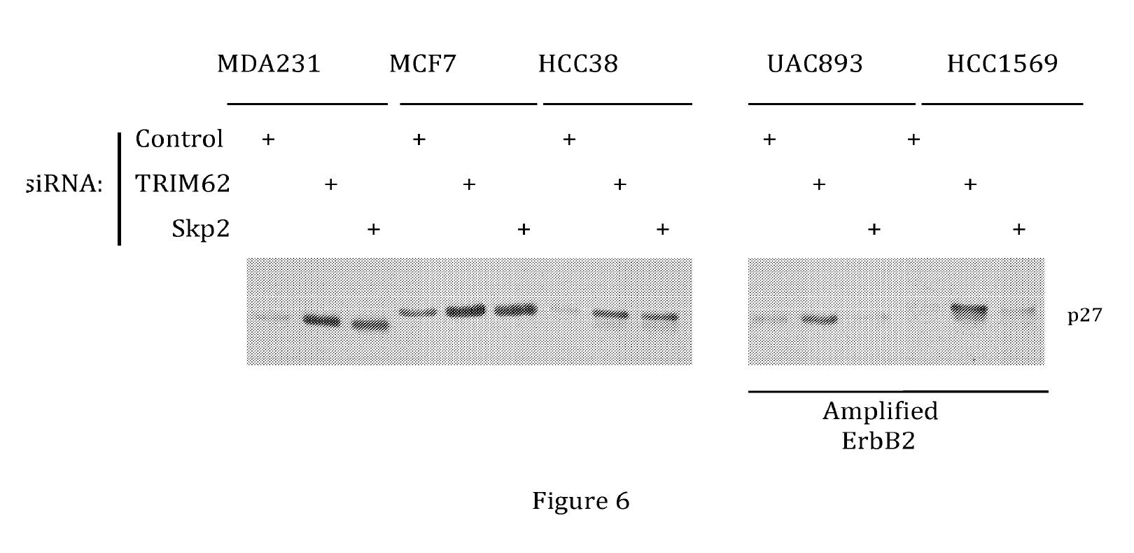

p27^{Kip1} and TRIM63 mRNA levels were measured in ErbB2 amplified breast cancer cells and non-ErbB2 amplified breast cancer cells. Results are shown in FIGS. 5A and B. mRNA levels were assessed in proliferating cells (gray bars) or quiescent cells (black bars). Results for breast cancer cell lines with amplified ErbB2 gene are boxed at the right side of the graphs. FIG. 5A shows levels of TRIM62 mRNA in breast cancer cells and FIG. 5B shows levels of p27^{Kip1} mRNA in breast cancer cells. Levels of both TRIM62 and p27^{Kip1} mRNAs were higher in ErbB2 amplified breast cancer cells as compared to non-ErbB2 amplified breast cancer cells.

TRIM62 activity regulates expression of p27^{Kip1} protein in many breast cancer cell lines. Levels of p27^{Kip1} were assayed in breast cancer cells with unmodified ErbB2 expression (MDA231, MCF7, and HCC38) and breast cancer cells with amplification of the ErbB2 gene (UAC893 and HCC1569) with the following additions: control siRNA, TRIM62 siRNA, and Skp2 siRNA. SKP2 is a known negative regulator of p27^{Kip1} levels. Levels of p27^{Kip1} protein were determined by Western blotting with a p27^{Kip1} specific antibody. In breast cancer cells with unmodified ErbB2 expression, p27^{Kip1} protein levels increased as compared to the control after addition of TRIM62 siRNA or Skp2 siRNA. In breast cancer cells with amplification of the ErbB2 gene, p27^{Kip1} protein levels increased as compared to the control after addition of TRIM62 siRNA. Addition of the Skp2 siRNA to breast cancer cells with an amplified ErbB2 gene did not affect the level of p27^{Kip1} protein. Subcellular localization of p27^{Kip1} protein in breast cancer cells with an amplified ErbB2 gene was investigated. p27^{Kip1} was detected using a p27^{Kip1} specific antibody. DAPI was used to identify cell nuclei. With addition of a control siRNA or a Skp2 siRNA, p27^{Kip1} protein was predominantly found in the cell cytoplasm, with a few instances of nuclear localization. Data not shown. In contrast, after addition of a TRIM62 siRNA, p27^{Kip1} protein was predominantly found in the nucleus. Data not shown.

### Example 3

### Inactivation of TRIM62 Enhances Effects of ErbB2 Antibodies on p27 Expression in ErbB2 Amplified Breast Cancer Cells

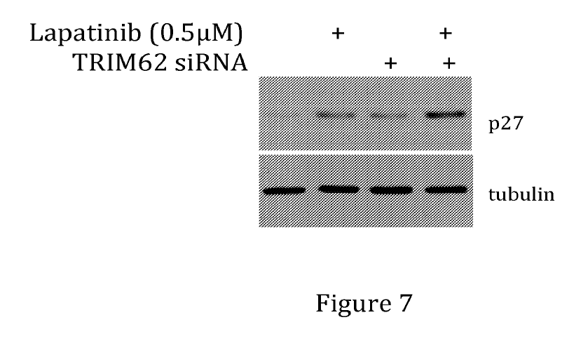

p27^{Kip1} protein levels were measured in breast cancer cells with ErbB2 gene amplification after administration of an ErbB2 specific mall organic molecule, lapatinib. p27^{Kip1} expression levels were higher after lapatinib was administered. Results are shown in FIG. 7. As before, expression of TRIM62 siRNA in the cells also increased the level of p27^{Kip1} protein expression. p27^{Kip1} protein levels were highest when lapatinib was administered to cells that expressed the TRIM62 siRNA. Results are shown in FIG. 7. The increased levels of p27^{Kip1} expression coincided with increased nuclear localization of p27^{Kip1}. Data not shown.

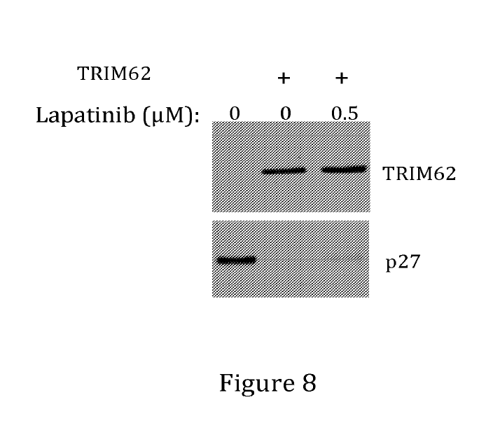

The effect of overexpression of TRIM62 protein on p27^{Kip1} expression was determined in breast cancer cells with ErbB2 gene amplification. FIG. 8 demonstrates that TRIM62 overexpression prevents lapatinib-dependent p27^{Kip1} accumulation in breast cancer cells with an amplified ErbB2 gene. A breast cancer cell line with an amplified ErbB2 gene (UACC893) was transfected with a TRIM62 expression vector or an empty vector. p27^{Kip1} levels were assayed by Western blotting using a p27^{Kip1} specific antibody. Cells were treated with 0.5 μM lapatinib or buffer. As compared to an empty vector control, cells that overexpressed TRIM62 had low levels of p27^{Kip1} protein expression. Administration of lapatinib in the presence of TRIM62 overexpression did not result in increased expression of p27^{Kip1} protein. Results are shown in FIG. 8.

### Example 4

### Assessment of the Effect of TRIM62siRNA on Normal Breast Cells and HER2 Positive Breast Cancer Cells

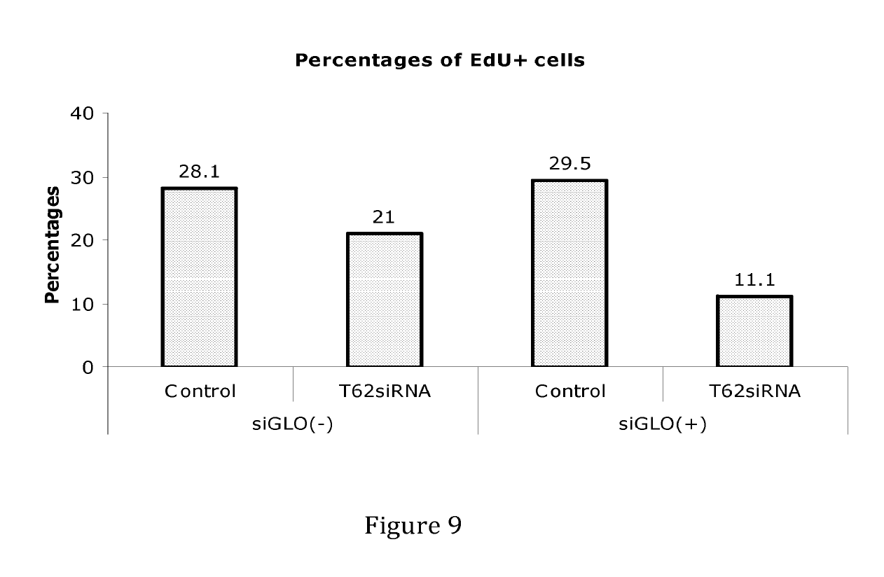

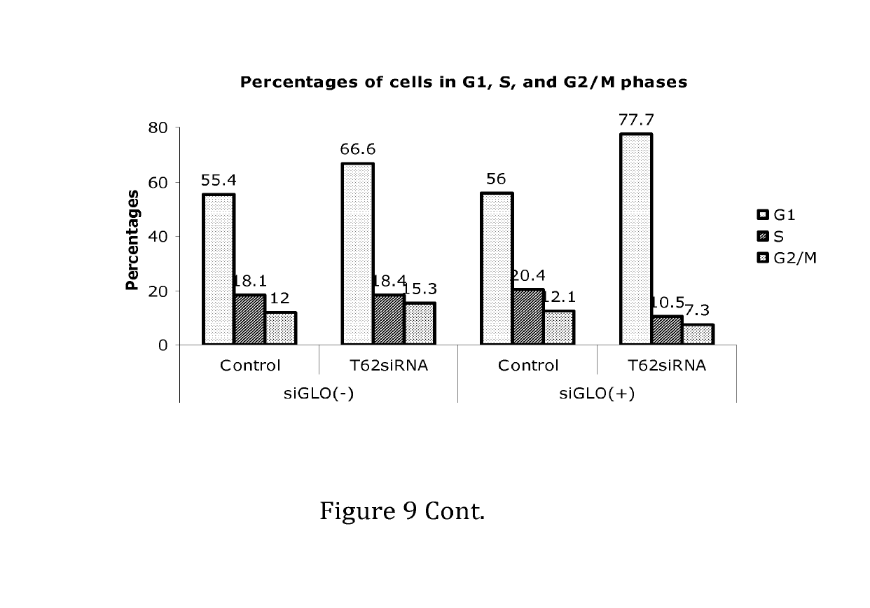

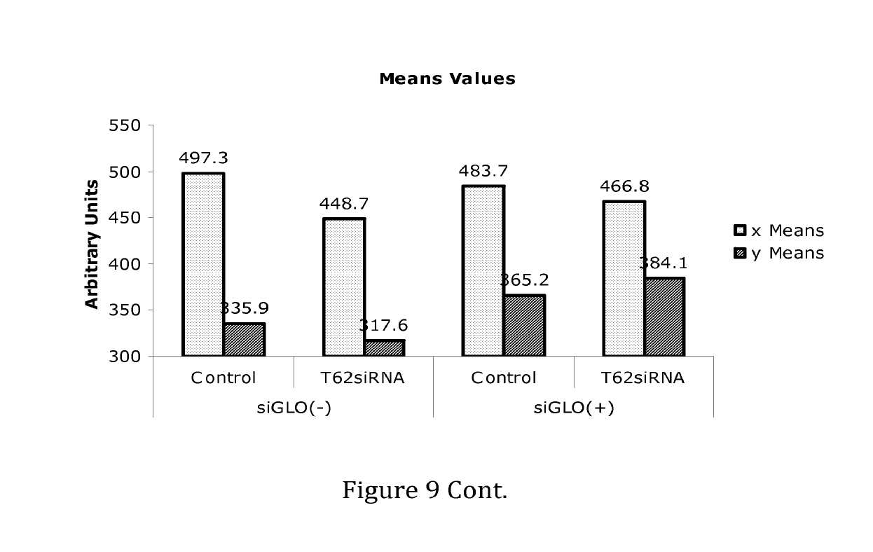

4×10⁵ MCF10A cells (immortalized, non-transformed epithelial cells derived from human fibrocystic mammary tissue; ATCC CRL-10217) or BT474 cells (Her2⁺ human breast cancer cell line) were seeded in growth media (DMEM/F12 (Gibco), 5% horse serum (Invitrogen), 1 mg/ml EGF (Peprotech), 1 mg/ml hydrocortisone (Sigma), 1 mg/ml cholera toxin (Sigma), 10 mg/ml insulin (Sigma), and Pen/Strep (Invitrogen)). After 24 hours, media was changed to antibiotic-free growth medium, and lipofectamine 2000 was used to cotransfect MCF10A cells with either 15 nM Trim62SiRNA (SEQ ID NO: 9; 5′ AAGAAGCAGAGAAGCGCGC-3″ (Integrated DNA Technologies)) and 15 nM siGlow (non-targeting control siRNA labeled with Cy3 (Thermo Scientific)), or 15 nM Trim62scrambled siRNA (SEQ ID NO:10; 5′ GCGAGGACGGACAAA CAGA 3′ (Sigma)) and 15 nM siGlow; for BT474 cells 25 nM siRNA was used. 6 hours post transfection, media was replaced with growth media containing antibiotics, and 48 hours postransfection, MCF10A cell were incubated for 1.5 hours with 10 nM EdU (5-ethynyl-2′-deoxyuridine, an analog of BrdU); for BT474 cells incubation with EdU was for 12 hours. Cells were trypsinized, washed with PBS-1% FBS, and sorted for Cy3 transfection marker using a cell sorter (BD FACSArisII cell sorter). Cells positive for Cy3 were fixed and permeablized overnight with 70% EtOH in PBS. Alexa fluoro 647 Click-it Flow Cytometry Assay Kit was used to label EdU, and propidium iodide (PI) was used to label DNA content and cell cycle distribution. A BD FACS Canto Flow Cytometer was used to measure 15,000 events using a 660 nm filter for EdU detection and a 610 nm filter for PI detection. The data was collected using a BD FACS Diva software package and analyzed using BD CellQuest Pro Software. The MCF10A cells were found to have 29.5% EdU⁺ cells when treated with the control SiRNA and 11.1% EdU⁺ cells with treated with T62siRNA. BT474 cells on the otherhand had 17.5% EtU⁺ cells when treated with Control siRNA and 9% EtU⁺ cells when treated with T62 siRNA confirming the antiproliferative effects of TRIM62 knowkdown by siRNA. FIG. 9 demonstrates the effect of TRIM62 siRNA on cell cycle progression.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

1. A method of treating a cancer in a human subject, the method comprising the step of administering to the human subject a therapeutically effective amount of an inhibitor of a tripartite motif-containing 62 (TRIM62) protein, wherein the TRIM62 protein has at least 95% identity to SEQ ID NO: 1 and wherein the TRIM62 protein regulates expression of a cyclin-dependent kinase inhibitor 1B (p27^{Kip1}) protein.

2. The method of claim 1, wherein the inhibitor of the TRIM62 protein is a member selected from the group consisting of an siRNA molecule, an antisense molecule, a small organic molecule, and an antibody that specifically binds to the TRIM62 protein.

3. The method of claim 1, wherein the cancer includes cells that overexpress an erythroblastic leukemia viral oncogene homolog 2 (ErbB2) protein.

4. The method of claim 3, further comprising a step of administering to the human subject an inhibitor of the ErbB2 protein.

5. The method of claim 4, wherein the inhibitor of the ErbB2 protein is selected from the group consisting of trastuzumab and lapatinib.

6. The method of claim 3, wherein the cancer is a member selected from the group consisting of breast cancer, ovarian cancer, prostate cancer, and gastric cancer.

7. The method of claim 1, wherein the inhibitor of the TRIM62 protein is a TRIM62 specific siRNA molecule.

8. The method of claim 7, wherein the siRNA molecule has a nucleic acid sequence selected from the group consisting of SEQ ID NO:5, 6, 7, and 8.

9. A method of identifying a compound that inhibits proliferation of a mammalian cell, the method comprising the steps of
i) contacting a tripartite motif-containing 62 (TRIM62) protein or a host cell comprising a TRIM62 protein with a test compound, wherein inhibition of an activity of the TRIM62 protein in the host cell results in increased expression of a cyclin-dependent kinase inhibitor 1B (p27^{Kip1}) protein; and
ii) assaying an activity of the TRIM62 protein or cellular expression of the TRIM62 protein in the presence of the test compound, wherein a difference in activity or expression in the presence of the test compound as compared to a control indicates that the compound modulates the activity or expression of the TRIM62 protein, thereby identifying the compound that inhibits proliferation of the mammalian cell.

10. The method of claim 9, wherein the test compound is a member selected from the group consisting of an siRNA, an antisense molecule, a small organic molecule, and an antibody that specifically binds to a TRIM62 protein.

11. The method of claim 9, wherein the activity of the TRIM62 protein or cellular expression of the TRIM62 protein is assayed in a mammalian cancer cell or an extract from a mammalian cancer cell.

12. The method of claim 11, further comprising the step of assaying an activity or cellular expression of the p27^{Kip1} protein in the mammalian cancer cell or the extract from the mammalian cancer cell.

13. The method of claim 11, wherein the mammalian cancer cell overexpresses an erythroblastic leukemia viral oncogene homolog 2 (ErbB2) protein.

14. The method of claim 13, further comprising a step of assaying activity of the TRIM62 protein or cellular expression of the TRIM62 protein in the presence of an inhibitor the ErbB2 protein.

15. A method of diagnosing a cancer with increased levels of expression of an epidermal growth factor receptor (EGFR) receptor family member, wherein the cancer is resistant to treatment with a compound that specifically inhibits activity of the EGFR receptor member, the method comprising the steps of
determining the level of expression or activity of a tripartite motif-containing 62 (TRIM62) protein in a sample from the cancer and comparing the TRIM62 protein expression or activity level to a control sample, wherein a difference from the control indicates that the cancer is resistant to treatment with a compound that specifically inhibits activity of the EGFR receptor family member.

16. The method of claim 15, wherein the level of TRIM62 protein is measured in the cancer sample and the control.

17. The method of claim 15, wherein the level of TRIM62 mRNA is measured in the cancer sample and the control.

18. The method of claim 15, further comprising a step of determining the level of expression of a cyclin-dependent kinase inhibitor 1B (p27^{Kip1}) protein in the cancer sample and the control, wherein a difference from the control indicates that the cancer is resistant to treatment with a compound that specifically inhibits activity of the EGFR receptor family member.

19. The method of claim 15, wherein the cancer is a breast cancer and the EGFR family member is an erythroblastic leukemia viral oncogene homolog 2 (ErbB2) protein.

20. The method of claim 19, wherein the compound that specifically inhibits activity of the ErbB2 protein is a member selected from the group consisting of lapatinib and trastuzumab.

21. A pharmaceutical composition comprising a modulator of a tripartite motif-containing 62 (TRIM62) protein, wherein the TRIM62 protein has at least 95% identity to SEQ ID NO: 1 and wherein the TRIM62 protein regulates expression of a cyclin-dependent kinase inhibitor 1B (p27^{Kip1}) protein.

22. The pharmaceutical composition of claim 21, wherein the modulator of the TRIM62 protein inhibits an activity of the TRIM62 protein or cellular expression of the TRIM62 protein.

23. The pharmaceutical composition of claim 22, wherein the modulator of the TRIM62 protein is an siRNA molecule that inhibits expression of the TRIM62 protein in a host cell.

24. The pharmaceutical composition of claim 23, wherein the siRNA molecule has a nucleic acid sequence selected from the group consisting of SEQ ID NO:5, 6, 7, and 8.

25. The pharmaceutical composition of claim 21, further comprising an inhibitor of an epidermal growth factor receptor (EGFR) receptor family member.

26. The pharmaceutical composition of claim 25, wherein the EGFR family member is a member selected from the group consisting of the epidermal growth factor receptor (EGFR), an erythroblastic leukemia viral oncogene homolog 2 (ErbB2), ErbB3, and ErbB4.

27. The pharmaceutical composition of claim 25, wherein the inhibitor of the EGFR family member is selected from the group consisting of trastuzumab lapatinib, gefitinib, erlotinib, cetuximab, panitumumab, pertuzumab, and canertinib.

28. The pharmaceutical composition of claim 25, wherein the EGFR family member is ErbB2 and the inhibitor of the EGFR family member is selected from the group consisting of trastuzumab and lapatinib.
